(19) United States
(12) Patent Application Publication
(10) Pub. No.: US 2010/0305658 A1
(43) Pub. Date: Dec. 2, 2010

(54) APPARATUS AND METHOD FOR MODULATING NEUROCHEMICAL LEVELS IN THE BRAIN

(75) Inventors: Kendall H. Lee, West Lebanon, NH (US); Charles D. Blaha, Germantown, TN (US)

Correspondence Address: Edwards Angell Palmer & Dodge LLP, 111 HUNTINGTON AVENUE, BOSTON, MA, 02199, US

(73) Assignees: Dartmouth College; University of Memphis

(21) Appl. No.: 12/749,925

(22) Filed: Mar. 30, 2010

Related U.S. Application Data
(63) Continuation of application No. 11/243,565, filed on Oct. 5, 2005, now abandoned.
(60) Provisional application No. 60/615,995, filed on Oct. 5, 2004.
(60) Provisional application No. 60/616,000, filed on Oct. 5, 2004.
(60) Provisional application No. 60/669,483, filed on Apr. 8, 2005.
(60) Provisional application No. 60/669,743, filed on Apr. 8, 2005.

(51) Int. Cl. A61N 1/36

(52) U.S. Cl. 607/45

(57) ABSTRACT

The present invention provides a method for modulating or regulating levels of a neurochemical in an individual using deep brain stimulation. More particularly, the invention relates to a method of treating neurological and psychiatric diseases by providing a feedback loop capable of maintaining central and/or peripheral nervous system neurochemical levels in an individual.

### RELATED APPLICATIONS

This application claims priority under 35 U.S.C. §119 to U.S. Provisional Application Ser. No. 60/615,995 filed Oct. 5, 2004, U.S. Provisional Application Ser. No. 60/616,000 filed Oct. 5, 2004, U.S. Provisional Application Ser. No. 60/669,743 filed Apr. 8, 2005, and U.S. Provisional Application Ser. No. 60/669,483 filed Apr. 8, 2005, the entirety of which are incorporated herein by reference.

### BACKGROUND OF THE INVENTION

Regulation of the levels of various neurochemicals and other chemicals in the central and peripheral nervous system is likely to provide a critical mechanism for the amelioration and/or prevention of neurodegenerative and psychiatric diseases in humans. While it is known in the art that electrical stimulation of deep brain structures is capable of treating the symptoms of some diseases such as Parkinson's disease (see, e.g., Benabid et al., 2000 Neurology, 55:s40-44), there has been a need in the art for methods of detection of levels of neurochemicals in hallmark diseases and use of this information to prevent the onset of the acute symptoms of the disease. While the ability to measure evoked release of neurochemicals in particular brain regions is known (see, e.g., Dugast et al., 1994 Neuroscience 62:647), the prior art, however, does not teach a method or device that utilizes such information to initiate treatment of an individual by deep brain stimulation (DBS).

### SUMMARY OF THE INVENTION

A principle feature of the present invention is to provide electrical stimulation applied to the central and/or peripheral nervous system of an individual using a deep brain stimulator (DBS) in response to the detection of a change in neurochemical levels in a particular region of the central/peripheral nervous system. Neurochemical refers to a chemical substance released from or which acts on neurons and/or glia during or as a result of neurotransmission or neurosecretion. Neurochemicals include, but are not limited to neurotransmitters, neuromodulators, neuropeptides, and/or neuroregulators. Exemplary neurochemicals include dopamine, acetylcholine, glutamate, norepinephrine, epinephrine, serotonin, and their precursors and metabolites (e.g., L-DOPA and DOPAC, respectively). The central nervous system may include, but is not limited to, structures in the brain (including the spinal cord) such as the thalamus, substantia nigra pars compacta and pars reticulata, cerebral cortex, caudate-putamen, globus pallidus, cerebellum, limbic structures, cranial nerve nuclei, and brain stem. The peripheral nervous system refers to peripheral ganglia of the somatic and/or autonomic nervous system, such as, but not limited to, spinal ganglia, enteric ganglia, and cardiac ganglia. The peripheral nervous system, as used herein, also refers to the target organs of the peripheral autonomic nervous system, including, but not limited to, the adrenal gland, carotid body, and smooth muscle. As used herein, the peripheral nervous system preferably does not include peripheral nerves.

The invention features a DBS device that includes a neurochemical sensor, a control module having electronic circuitry capable of determining whether an amount of neurochemical is different from a predetermined amount, and a stimulation module under the control of the control module. The sensor is used to measure the amount of neurochemical in a particular region of the central/peripheral nervous system, and that information is relayed to the control module. The sensor may also be adapted to measure the levels of neurochemicals introduced to the central and/or peripheral nervous system. The sensor may be any sensor that permits the measurement of neurochemicals in vivo, including, but not limited to sensors that may be used in microdialysis, constant potential amperometry, fast-scan cyclic voltammetry, high-speed chronoamperometry, differential normal-pulse voltammetry, or any number of electroanalytical techniques known in the art. If the amount of neurochemical measured by the sensor is different from a desired amount, a signal indicative thereof is sent to the stimulation module. The stimulation module then generates an electrical signal that is transmitted to the central and/or peripheral nervous system of the individual.

The present invention also provides a method for modulating selected neurochemical levels in the central and/or peripheral nervous system of an individual. A sensor, capable of detecting the levels (or changes in levels) of extracellular concentrations of a neurochemical, is placed in a region of the central and/or peripheral nervous system of an individual. The sensor is directly or indirectly connected to a control module which can determine if the amount of neurochemical measured using the sensor is different from a desired amount. Levels of neurochemicals are measured, and a difference, if any, in the level of neurochemical relative to a desired amount is detected. Upon detection of a difference in the level of neurochemical relative to the desired amount, a signal indicative thereof is sent from the control module to a stimulation module. A stimulation electrode, directly or indirectly connected to a stimulation module, is placed in or on the central and/or peripheral nervous system of the individual. Electrical stimulation is generated by the stimulation module and transmitted to the central and/or peripheral nervous system of the individual by way of the stimulation electrode.

Using the methods outlined above, the present invention also features a method for treatment of neurological and psychiatric disorders such as Parkinson's disease, tremor, epilepsy, and depression in which DBS has been shown to be efficacious (see e.g., Diamond and Jankovic, J Neurol Neurosurg Psychiatry, 76:1188-1193, 2005; Benabid, Cur. Opin. Neurobio. 13: 696-706, 2003; Vonck et al., Epilepsia. 46 Suppl 5:98-99, 2005; Mayberg et al., Neuron, 45:651-660, 2005). This may also include other psychiatric and psychological disorders such as bipolar disorder, obesity, anxiety, drug abuse, post traumatic stress disorder, and schizophrenia.

The sensor useful in these methods can be any electrochemical sensor, such as but not limited to a carbon fiber electrode or other electrochemical sensors known in the art.

Other suitable sensors may be used, provided that they are able to detect extracellular levels of neurochemical in the central and peripheral nervous system. Sensors and electronic circuitry may be adapted to perform constant-potential amperometry, fast-scan cyclic voltammetry, high-speed chronoamperometry, differential normal-pulse voltammetry, or any number of electroanalytical techniques. The sensor and stimulation electrode may be placed in the same region or different regions of the individual's central and/or peripheral nervous system. In any of the embodiments of the invention, the stimulation electrode and neurochemical sensor may be present on a single probe.

The stimulation electrode may be a single electrode, or a plurality of electrodes, provided that each of the plurality of electrodes is directly or indirectly connected to the control module. The neurochemical sensor may also be a single sensor, or plurality of sensors and may be placed in any region of human nervous system in which the level of neurochemical is to be measured, including, but not limited to the central and peripheral nervous system.

The sensor may be adapted to measure the release and/or electrolysis (e.g., oxidation or reduction) of endogenous brain chemicals such as neurotransmitters and/or neuromodulators and neuroregulators including, but not limited to dopamine, acetylcholine, glutamtate, norepinephrine, epinephrine, serotonin, and their precursors and metabolites (e.g., L-DOPA and DOPAC, respectively). The sensor may also be adapted to measure the presence or activity of exogenous chemicals introduced to the central and peripheral nervous system.

The stimulation electrode may be placed in the central or peripheral nervous system brain regions such as, but not limited to the diencephalon, subthalamic nucleus (STN), medial forebrain bundle (MFB), nigrostriatal tract, or substantia nigra (SN), dorsal longitudinal fasciculus, hypothalamus, habenula, globus pallidus and pedunculopontine. Preferably, the stimulation electrode is placed in either or both of the STN and MFB.

The DBS described above may optionally include a chemical delivery module connected directly or indirectly to the control module. The methods of the invention may therefore include delivery of a compound by the chemical delivery module in response to a signal from the control module. Compounds useful for administration include, but are not limited to neurotransmitters, neuropeptides, neuromodulators, neuroregulators, receptor agonists, receptor antagonists, ion channel blockers, ion channel activators, and calcium chelators. These compounds are preferably a neurotransmitter such as dopamine, acetylcholine, glutamate, norepinephrine, epinephrine, histamine, serotonin, neuropeptides (such as cholecystokinin) and their precursors and metabolites (e.g., L-DOPA and DOPAC, respectively).

The present invention provides a method for positioning a stimulation electrode in a brain of an individual for electrical stimulation and neurochemical recordings in the central nervous system. The neurochemical sensor, capable of detecting extracellular concentrations of neurochemicals evoked (e.g., released or modulated) by electrical stimulation, is placed in a first brain region of an individual. The neurochemical sensor is directly or indirectly connected to a control module that can determine if the amount of neurochemical measured using the sensor reaches a predetermined amount (that is, is at least at a predetermined amount, or is approximately at a predetermined amount). A stimulation electrode, directly or indirectly connected to a stimulation module is placed in a second brain region or structure of the individual. Electrical stimulation is generated by the stimulation module and transmitted to the brain of the individual by way of the stimulation electrode. Neurochemical levels are measured by the sensor, and a determination is made by the control module as to whether the amount of neurochemical in the first brain region reaches a predetermined amount. If the amount of neurochemical measured in response to the electrical stimulation does not reach the predetermined level, the positioning of the stimulation electrode is changed, and the steps of electrical stimulation, neurochemical measurement, comparison to a predetermined amount, and stimulation electrode repositioning are repeated until the predetermined amount of neurochemical release is reached. Alternatively, or in addition, the repositioning of the stimulation electrode and subsequent measurement of neurochemical levels may be repeated over a predetermined or randomized series of stimulation electrode positions, and the level of neurochemical measured in each position compared to determine the position in which electrical stimulation elicited the largest or least amount of neurochemical extracellular concentration. This may then be chosen as the site of stimulation of electrode placement. Similarly, the positioning and repositioning of the stimulation electrode and measurement of neurochemical may be repeated until a first stimulation electrode position is reached where the amount of neurochemical reaches or exceeds a predetermined level.

In any of the foregoing embodiments of the invention, the stimulation electrode and sensor may be present on a single probe.

### BRIEF DESCRIPTION OF THE FIGURES

FIG. 1 shows a flowchart of a feedback circuit of the present invention that may be used to modulate neurochemical levels in an individual.

FIG. 2 shows a flowchart of a more detailed feedback circuit of the present invention that may be used to modulate neurochemical levels in an individual.

FIG. 3 shows a block diagram of a deep brain stimulator useful in the present invention.

FIG. 4 shows a more detailed block diagram of the deep brain stimulator of the invention that shows additional components that may be included in the deep brain stimulator.

FIG. 5 shows extracellular recording of an subthalamic nucleus (STN) neuron before, during, and after high frequency stimulation (HFS) of the STN (100 Hz).

FIG. 6 shows a schematic drawing of the rat brain with a stimulating electrode placed in the STN and an amperometry electrode placed in the striatum. FIG. 6 also shows changes in dopamine oxidation currents in response to STN stimulation.

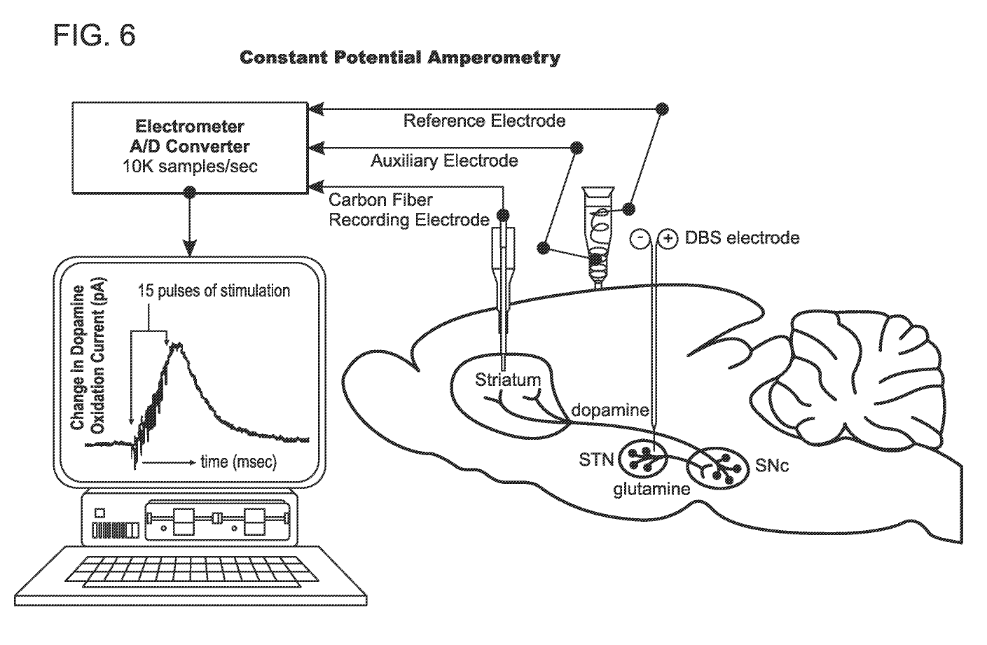

FIG. 7 shows changes in dopamine oxidation currents in response to STN stimulation in the presence of nomifensine (FIG. 7a) or desipramine and fluoxetine (FIG. 7b).

FIG. 8 shows frequency and intensity dependence of STN stimulation-induced changes in dopamine oxidation current.

FIG. 9 shows a photomicrograph of a coronal section of the ferret STN stained with a mono-clonal antibody to the dopamine transporter (DAT).

FIG. 10 shows differential changes in dopamine oxidation currents following stimulation of the STN vs. the white matter dorsal to the STN.

FIG. 11 shows a more detailed example of a deep brain stimulator of the invention.

FIG. 12 is a schematic drawing showing an example of a single probe comprising electrical stimulation electrodes and constant potential amperometry (neurochemical sensor) electrodes.

FIG. 13 shows subthalamic nucleus (STTN) and ventrolateral (VL) thalamic glutamate release with high frequency stimulation (HFS) in the rat in vivo.

FIG. 14 shows HFS is able to block network oscillations.

FIG. 15 depicts extracellular recordings from lamina A1 of the ferret LGN slice with GABA_{A} antagonist picrotoxin (20 μM) in bath.

FIG. 16 shows HFS in the ferret thalamic slice results in glutamate release that is not blocked by the classic neuronal exocytosis inhibitors, TTX or low Ca++, high Mg++ bath solution.

FIG. 17 shows GFAP staining and Glutamate release in primary astrocytic cultures.

### DETAILED DESCRIPTION

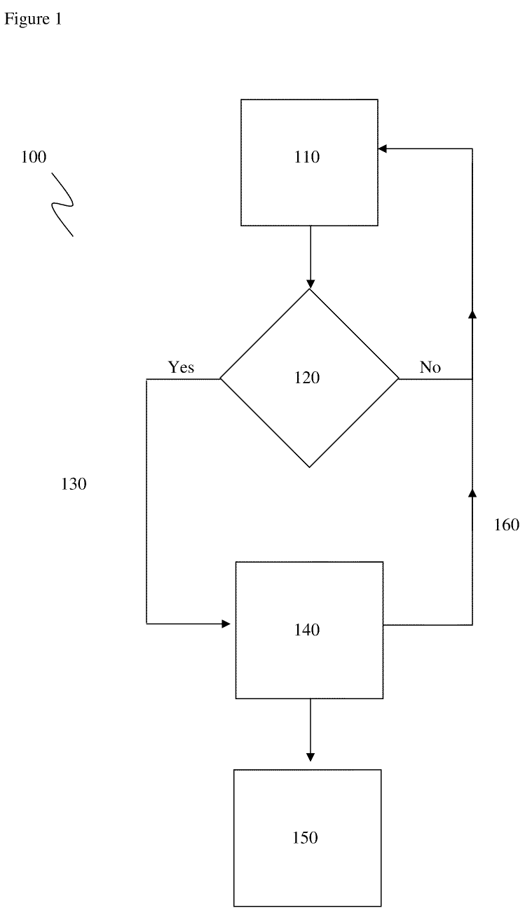

The present invention provides a method and apparatus for the detection, monitoring, and regulation of levels of selected neurochemicals in the central and peripheral nervous system. The invention features a mechanism for detecting the level of a neurochemical, including a change in a neurochemical level, indicative of the onset of a neurological and/or psychiatric condition, or symptom thereof, where the condition is associated with alterations in the level of a neurochemical. Electrical stimulation is then provided to the brain in response to treat the condition. DBS is a viable treatment alternative for patients with Parkinson's disease, essential tremor, dystonia, cerebellar outflow tremors and depression. For example, electrical stimulation of the STN and/or medial forebrain bundle (MFB) is used to treat symptoms of Parkinson's disease. Electrical stimulation of the STN and adjacent brain structures increase striatal (caudate-putamen) dopamine release which can serve as a neurochemical feedback signal to determine the efficacy of stimulation. Additional examples include electrical stimulation to the subgenual cingulate region and temporal cortical regions of the brain for the treatment of depression and epilepsy, respectively. In these latter cases, on-line monitoring of the local release of a neurochemical, such as norepinephrine and glutamate can also serve as a neurochemical feedback signal to determine the efficacy of stimulation. FIG. 1 shows a general feedback system 100 of the invention, which is used to modulate or regulate levels of a neurochemical in an individual and to treat or prevent the onset of neurodegenerative disease with attendant alterations in levels of a neurochemical, or symptoms thereof. In a first step 110, the amount of neurochemical in a first brain region or peripheral nervous system structure of an individual is determined. Methods for measuring neurochemical levels in vivo are known in the art and may be readily adapted to function in the present invention. For example, neurochemical levels may be measured using microdialysis and various electroanalytical techniques including constant-potential amperometry, fast-scan cyclic voltammetry, high-speed chronoamperometry, and differential normal-pulse voltammetry. Microdialysis is one of the most widely utilized in vivo methods for measuring neurochemical release in an animal. Briefly, a dialysis probe that is permeable to small molecules is placed in the brain and perfused with an artificial cerebrospinal fluid. Molecules of appropriate size will diffuse into the probe, and are collected and analyzed outside the animal (e.g., after separation by HPLC). Microdialysis can provide a high degree of chemical selectivity and sensitivity, but offers relatively poor temporal resolution (see, e.g., Lu et al., 1998 J. Neurochem. 70:584; Peters and Michael, 1998 J. Neurochem. 70:594).

In addition to microdialysis, other electroanalytical techniques can be used to measure the level of a neurochemical. Constant potential amperometry is an electroanalytical technique in which an electrical potential that is able to oxidize or reduce a subject molecule is applied to an electrode placed in a particular brain region of an individual. The amperometry electrode can record the current generated by the oxidation or reduction of a neurochemical. The amperometry electrode is able to record quickly neurochemical concentrations and yields high temporal resolution (see, e.g., Dugast et al., supra). Fast-scan cyclic voltammetry methods are described in, for example, Stamford et al., 1995 In: Boulton et al., Eds. Neuromethods: voltammetric methods in brain systems v. 27 Totowa, N.J.: Humana Press: 81-116. Chronoamperometry is described in, for example, Blaha and Phillips, 1996 Behavioural Pharmacology 7:675-708; Hoffman and Gerhardt, 1999 J. Phramacol. Exp. Ther. 289:455. Differential normal-pulse voltammetry is described, for example, in Mas et al., 1990 Neurosci. Lett. 110:303. Levels of neurotransmitters can also be measured in the peripheral nervous system. For example, Mermet et al., 1990 Acta Physiol Scand. 140:323-329 have shown the applicability of on-line electrochemical techniques to monitoring norepinephrine release in smooth muscle evoked by electrical stimulation of the sympathetic nerves. Hentall and Sagen, 2000 Neurosci Lett. 286:95-98 have electrochemically recorded norepinephrine release from chromaffin cells. Clark and Ewing, 1997 Mol Neurobiol. 1997 15:1-16 have reviewed the applicability of electrochemical monitoring of catecholamine levels in superior cervical ganglion neurons and pancreatic beta-cells. Rigual et al., 2000 Pflugers Arch. 439:463-470 have electrochemically monitored catecholamine release in the carotid body and Niederhoffer et al., 2001 Br J Pharmacol. 134:1319-1327 have similarly recorded epinephrine release from adrenal medullary cells. Thus, one of skill in the art will recognize that any method that permits the measurement of neurochemicals in vivo may be used according to the invention to carry out step 110.

Step 110 of method 100 measures the amount of a neurochemical in a particular brain area. This is accomplished either by measuring the amount of the neurochemical using microdialysis or by measuring the electrolysis of the neurochemical, for example, by electro-oxidizing the neurochemical and measuring the resulting oxidation current using an electroanalytical technique such as constant potential amperometry. The present invention contemplates that any neurochemical that may be oxidized or reduced can be measured. Such neurochemicals include, but are not limited to dopamine, acetylcholine, glutamate, norepinephrine, epinephrine, serotonin, and their precursors and metabolites (e.g., L-DOPA and DOPAC, respectively). The specific parameters used for constant potential amperometry (or other electroanalytical technique) may be adjusted by one of skill in the art according to the particular oxidation/reduction potential of a given neurochemical. As used herein, “amount of a neurochemical” will refer to either or both of measurement of actual neurochemical levels (e.g., by microdialysis) or measurement of oxidation and reduction currents proportional to neurochemical levels as produced by the electrolysis of a neurochemical.

Neurochemical extracellular levels in an individual may be measured in step 110 from any brain area of interest. In particular, neurochemicals may be measured in the cortex, striatum, cerebellum, hippocampus, and other nuclei and subnuclei known to those of skill in the art. Preferably, the amount of a neurochemical is measured from basal ganglia structures such as the caudate-putamen (striatum in lower mammals such as the rat).

In second step 120, the amount of a neurochemical measured is compared to a predetermined value to see if there is a difference in the amount of the neurochemical measured from a particular brain area. The predetermined value used in step 120 will differ depending on the particular neurochemical being examined. The predetermined value in step 120 may be a permanently fixed value, or may be manipulated by an individual or a physician in a manner dependent on the individual, the neurochemical to be measured, or any attendant pathology in the individual. For example, where an individual has a pathology which causes a reduced level of a given neurochemical, the predetermined level used in step 120 may be lower than in an individual that does not have the pathology. Step 120 may be adapted to determine whether there is any change in the amount of neurochemical measured in step 110, or may be adapted to determine whether the change in the amount of neurochemical measured differs from the predetermined amount by a certain threshold, for example, will identify a change in neurochemical levels that are at least 5%, 10%, 15%, 20%, 30%, or 40% or more different from the predetermined amount.

Detecting a change in the amount of neurochemical measured in a given brain region may be performed using hardware, software, or firmware. For example, the detection of a change in neurochemical levels may be performed using hardware such as a gating or filtering circuit that only permits the transmission of signals indicating that the neurochemical levels detected in step 110 have specified characteristics, such as being at least 10% different from the predetermined amount in step 120. Such circuits are well known in the art. Alternatively, step 120 may utilize a processor programmed with firmware or software to analyze the neurochemical levels detected in step 110 to identify changes in the amounts measured. If no change in neurochemical level is detected in step 120, then steps 110 and 120 are repeated. If a change in neurochemical level is detected in step 120, then a signal is sent from the processor used in step 120 to a stimulation module (step 130). The stimulation module is capable of delivering electrical stimulation to the brain of the individual, either directly, or indirectly. For example, the signal may also trigger administration of a neuroactive compound before, after, or coincident with the electrical stimulation provided by the stimulator. In step 140, the stimulation module generates an electrical signal having the parameters outlined in, but not limited to, those shown in Table 1. Preferably, the stimulation module provides electrical stimulation having an amplitude of between 200 and 600 μA, and a frequency of between 35 and 150 Hz. More preferably, the stimulation module generates electrical stimulation having an amplitude of 300 μA and a frequency of 50 Hz. The specific stimulation parameters may be modified by one of skill in the art to meet a particular application without departing from the scope of spirit of the invention.

TABLE 1
Stimulation Parameters

| Output | | | | |
|---|---|---|---|---|
| Voltage | Amplitude | Pulse Width | Frequency | Duration |
| 1-20 V | 10-1,600 μA | 50-500 μsec | 5-300 Hz | 0.001-60 s |

In step 150, the electrical stimulation generated in step 140 is applied to the brain of the individual to be treated. The electrical stimulation may be applied directly to particular brain regions such as the thalamus, STN, MFB, substantia nigra (SN), and/or nigrostriatal tract, or alternatively or in addition, the stimulation may be applied to afferent or efferent nigrostriatal fiber tracts, or other cortical or subcortical regions that are interconnected with the caudate-putamen. Following stimulation, a signal is sent back to the control module to reset the system (step 160) and start further measurements of neurochemical levels.

The method shown in FIG. 1 may be carried out using any of the deep brain stimulation systems described herein. Modification of the above described method to conform to particular aspects of an individual are within the scope of the invention, and the method may be readily adapted by one of skill in the art.

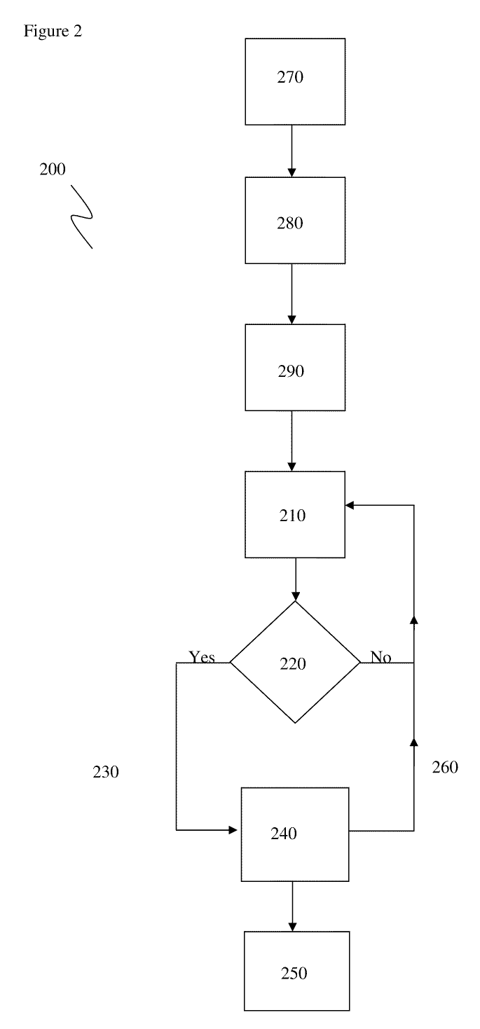

FIG. 2 shows a more detailed flow chart depicting method 200 that minors the steps of method 100, but includes additional steps 270-290 for measuring the amount of a neurochemical in an individual. In step 270, a deep brain stimulation system of the invention, including minimally a neurochemical sensor, a control module, and a stimulation module, is provided for measuring selected neurochemical levels in an individual. In step 280, the sensor is placed in or on the brain of an individual in which neurochemical levels are to be measured. The neurochemical sensor may be placed in any brain region in which neurochemical levels are to be measured, for example in the cortex, cerebellum, hippocampus, and any nuclei or subnuclei thereof, most preferably, in the caudate-putamen. In step 290, a stimulation electrode, directly or indirectly connected to the stimulation module, is placed in a brain region of the individual. The stimulation electrode may be placed in the same location as the sensor, or may be placed in a different location in the brain from the sensor. For example, the stimulation electrode may be placed in the cortex, thalamus, STN, MFB, SN, and/or nigrostriatal tract. One of skill in the art will recognize that the specific placement of the sensor and stimulation electrode will depend on the particular neurochemical that is to be measured, or the particular disease state that is to be treated. Steps 210-260 of method 200 are analogous to steps 110-160 of method 100, and will thus not be repeated.

The present invention also relates to a method for positioning a stimulation electrode in the brain of an individual. When used for treatment of neurological disease, such as Parkinson's disease it is necessary to position the stimulation electrode in the correct brain region. Correct positioning of the electrode may be guided by amperometric measurements of neurochemical release in response to stimulation of brain regions by the stimulation electrode. For example, if the stimulation electrode is to be placed in the subthalamic nucleus, the electrode can be first positioned using stereotactic coordinates, or other surgical procedures consistent with the standard of care used for intracerebral electrode placement. In addition, a sensor, such as a constant potential amperometry sensor, may be placed in or near, for example, the striatum. If correctly placed, electrical stimulation by the stimulation electrode should elicit some minimum amount of neurochemical release in the striatum. The amount of neurochemical release can be measured in the striatum and a determination made as to whether the amount measured reaches a predetermined minimum amount. If the amount of neurochemical release does not reach the predetermined amount, the stimulation electrode is repositioned and electrical stimulation is reapplied. This process can be repeated until a positioning of the stimulation electrode is achieved in which the amount of neurochemical release measured following electrical stimulation reaches or exceeds the predetermined amount of neurochemical release. Alternatively, the process of stimulation electrode positioning, electrical stimulation and neurochemical measurement may be repeated over a predetermined or random series of stimulation electrode positions. The levels of evoked neurochemical release in each position may be compared to select the proper stimulation electrode position (i.e., the position in which electrical stimulation evoked the greatest amount of neurochemical release).

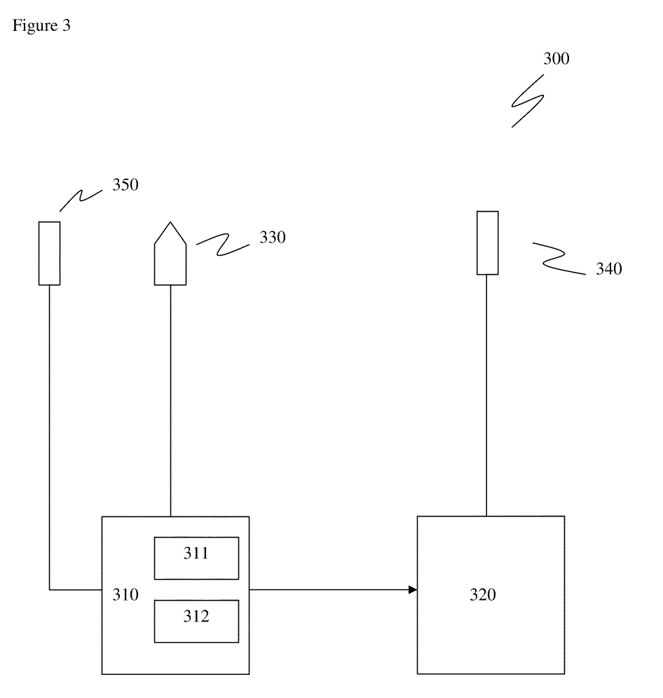

The present invention also relates to a deep brain stimulation system for providing electrical stimulation to the brain, or specific brain regions, of an individual in response to the detection of particular neurochemical levels in the caudate-putamen or other brain regions as described herein. FIG. 3 shows a block diagram of a deep brain stimulator system that may be used to perform the methods described herein. The deep brain stimulator 300, in its simplest form, includes a sensor 330, control module 310, stimulation module 320, stimulating electrode 340, and, optionally, reference/auxiliary combination electrode 350.

Sensor 330 may be any neurochemical sensor that is able to measure extracellular levels or a neurochemical in an individual. Exemplary forms of sensor 330 are a microdialysis probe that permits diffusion of neurochemical into the probe that is then analyzed off-line, for example by HPLC, to determine the amounts of neurochemical measured in a particular brain region, or an electrochemical sensor that is able to perform electrochemical detection methods on-line such as, but not limited to constant-potential amperometry, fast-scan cyclic voltammetry, high-speed chronoamperometry, and differential normal-pulse voltammetry. These methods are known in the art and are described above. An example of a neurochemical sensor 330 which may be used in the invention to perform the electrochemical detection methods described herein is a single- or multi-carbon fiber microelectrode (See, e.g., Yavich and Tiihonen, 2000 J. Neurosci. Meth. 104:55). Carbon fiber electrodes useful as a sensor 330 may be obtained commercially or may be fabricated by methods known in the art. For example, carbon fiber electrodes may be fabricated by threading a single or multiple number of carbon fibers (10 μm outer diameter) through a borosilicate glass capillary tube (WPI, Sarasota, Fla.) which is then heat pulled using a micropipette puller (e.g., P-91 puller, Sutter Instruments, Novato, Calif.) to form a tip through which the carbon fiber protrudes. The tip is then sealed with cyanoacrylate (i.e., super glue) and is allowed to set overnight. Carbon paste is then packed into the bore of the electrode, and a copper wire inserted to make contact with the carbon fiber. The copper wire is secured in place with super glue and the tip of the fiber is broken back under a microscope to create an active recording surface approximately 500 μm long. A multi-carbon fiber rod electrode, for example, may also serve as a neurochemical sensor and may be fabricated from a single vinylester-coated carbon rod (part number AE001115, GraphiteStore.com, Inc., Buffalo Grove, Ill.) that is machine-sanded at the tip of the rod to form a 1-2 mm length cone-shaped active recording tip. Electrical contact with the electrode can be made via an Amphenol pin (male or female) clamped to the opposing end of the rod. Additional methods of fabricating a neurochemical sensor are noted below.

Sensor 330 may be adapted for permanent placement in the brain of an individual, or may be placed temporarily in the brain of the individual, e.g., sensor 330 may be replaced after a given period of time with a new sensor. It will be understood that in some versions of sensor 330, the sensor is intended to be used in concert with the reference/auxiliary electrode 350. That is, if sensor 330 is used in, for example, constant-potential amperometry, sensor 330 is held at a constant potential versus the reference electrode component of 350. Reference/auxiliary electrodes 350 useful in the invention are known in the art and may consist of a standard silver-silver chlorided extracellular electrode (reference) and a stainless steel screw (auxiliary) placed in the skull of an individual such that the tip of the screw is in contact with the surface of the brain of the individual or as a single ring electrode fixed on the shaft of the sensor. The reference/auxiliary electrode 350 is directly or indirectly connected to control module 310.

Control module 310 includes electronic circuitry adapted to receive signals from sensor 330 (and in some cases to also receive signals from the reference electrode component of 350 and transmit signals to the auxiliary electrode component of 350) and determine the amount of neurochemical relative to a predetermined amount. Control module 310 optionally includes electrometer 311 for providing, in the case of constant potential amperometery, a constant potential to sensor 330 that detects the oxidation or reduction of neurochemicals in the vicinity of sensor 330. For example, oxidation of a chemical occurring at sensor 330 is detected as an oxidation current, which is then conveyed to control module 310. The oxidation current detected by sensor 330 is conveyed to the electrochemical processor 312 that carries out a processing such as step 120 in FIG. 1 or step 220 of FIG. 2. Electrochemical processor 312 receives signals from sensor 330 and determines whether the amount of neurochemical measured is different from a predetermined amount. If so, a signal is sent to stimulator module 320. The circuitry of electrochemical processor 312 may be a gating or filter circuit, which is designed to only allow electrical signals having set properties trigger a control signal to, for example, the high frequency stimulation module 320. Circuits of this type are known in the art and may be readily adapted for use in the instant invention. Alternatively, electrochemical processor 312 may comprise other hardware, firmware, or software, or may be a processor analogous to a general purpose computer programmed to perform the detection step 130 of FIG. 1. Parameters for detection of changes in neurochemical levels by electrochemical processor 312, which include, for example, predetermined values of a neurochemicals against which the actual measured levels of the neurochemical are compared, may be programmed as a permanent setting, or may be adjustable by either the individual, or by a physician treating the individual. Any data processed by electrochemical processor 312, or created as a result of such processing, may be optionally stored as memory as is conventional in the art. For example, such data may be stored in a temporary memory such as in a data buffer of the electrochemical device itself or the RAM of a given computer system or subsystem. In addition, or in the alternative, such data may be stored in longer-term storage devices, for example, magnetic disks, rewritable optical disks, and the like. For purposes of the disclosure herein, a computer-readable media may comprise any form of data storage mechanism, including such existing memory technologies as well as hardware or circuit representations of such structures and of such data.

Control module 310 is directly or indirectly connected to stimulation module 320, which, in turn, is capable of generating electrical signals having the properties outlined in, but not limited to, those shown in Table 1. Preferably, stimulation module 320 generates an electrical signal at 35 Hz or greater, 50 Hz or greater, 60 Hz or greater, 70 Hz or greater, and up to 100 Hz or greater. Stimulation module 320 preferably provides electrical stimulation at 200 μA or greater, 300 μA or greater, 600 μA or greater, and up to 800 μA or greater. More preferably, the stimulation module provides electrical stimulation at 50 Hz and/or 300 μA. Stimulation module 320 is directly or indirectly connected to stimulation electrode 340. An electrical signal generated by stimulation module 320 is conveyed to stimulation electrode 340 and thus directed into and/or onto the brain of the individual being treated. Stimulation electrode 340 may be any conductive electrode that is capable of delivering an electrical stimulus to brain tissue of an individual. Stimulation electrode 340 can include surface electrodes which may be removably placed on the scalp of the individual, and/or coaxial or other suitable electrodes as described below which are placed directly in the brain of an individual to be treated. While sensor 330 and electrode 340 generally must be located in or on the individual to be treated (particularly, in or on the brain of the individual), the other components of deep brain stimulator 300 may be located externally. For example, control module 310 and stimulation module 320 may be removably attached to the individual (e.g., by a belt clip, harness, or lanyard), or alternatively, may be miniaturized to suitable size for implantation in an individual (e.g., implanted under the skin in the abdomen, chest, or neck). Control module 310 and stimulation module 320 may be connected to each other and to the sensor and stimulation electrode 330 and 340 by suitable means known to those of skill in the art. These include, but not limited to wire, coaxial cable, optical cable, fiber optics, or infrared signals. Control module 310 and stimulation module 320 may be remote from one another, or control module 310 and stimulation module 320 may be incorporated into the same device by way of a housing, case, shell, frame, or other suitable mechanism, or packaging. One or more elements of the deep brain stimulator 300 may be permanently connected, for example, control module 310 and high frequency stimulation module 320 may be contained within a housing or other confinement and permanently connected by solder or other electrically conductive weld.

It will be appreciated by one of skill in the art that sensor 330 may be adapted to function both as sensor 330 and stimulation electrode 340. In this case, the electronic circuitry of control module 310 is further modified to include the capability to switch between (1) providing constant potential to and receiving oxidation and/or reduction signals from combined sensor/stimulator 330/340, and (2) providing electrical stimulation produced by stimulation module 320.

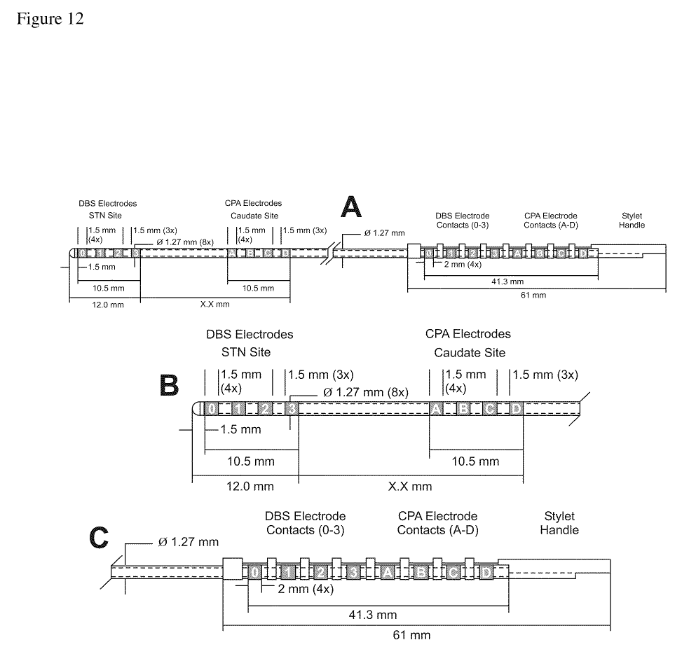

The stimulation electrode and sensor (e.g., constant potential amperometry sensor) may be further adapted to be included on a single probe for implantation in the brain of an individual. FIG. 12 shows a depiction of a combined deep brain stimulation electrode (DBS) and constant potential amperometric sensor (CPA) on a single probe. FIG. 12A is a full view of the probe showing the DBS electrodes and CPA electrodes as well as the stimulation electrode contacts and sensor contacts (i.e., where connection is made to the other components of the deep brain stimulator of the invention). Stimulation electrodes labeled 0-3 comprise four individual platinum-iridium ring electrodes for electrical stimulation of brain tissue in, for example, the subthalamic nucleus (STN). Although FIG. 12A shows four stimulation electrodes, the number of stimulation electrodes may be as few as one, or more than four. It will be appreciated by one of skill in the art this embodiment of the invention is not limited to the use of platinum-iridium for the stimulation electrodes, but that other conductive materials may be used within the scope of the invention.

As shown, constant potential amperometric electrodes labeled A-D comprise four individual carbon ring electrodes for monitoring extracellular neurochemical levels in brain tissue (for example, in the caudate nucleus). Although FIG. 12A shows four CPA electrodes, the number of CPA electrodes may be as few as one, or more than four. One of these electrodes may serve as an auxiliary/reference electrode. DBS and CPA electrode contacts labeled 0-3 and A-D, respectively, permit individual electrical contact with the deep brain stimulator of the invention. Although FIG. 12 shows the same number of DBS and CPA electrodes on a given probe, it will be understood by one of skill in the art that the respective numbers of DBS and CPA electrodes may vary relative to one another. The stylet handle permits permanent connection of the probe with a chronically implanted deep brain stimulator. The distances between components of the combined DBS and CPA probe shown in FIG. 12 are for example only, and may be modified as needed for a particular individual or application. The distance X.X mm separating the DBS and CPA electrodes on the shaft of the probe is a variable distance, and will ultimately correspond to the specific dorsal-ventral or medial-lateral distance separating the brain structures to be stimulated and recorded. FIGS. 12B and 12C are depictions of the same probe shown in FIG. 12A, but expanded in size for clarity of the component parts of the probe.

In addition to the components described above, control module 310 may optionally include conventional peripherals, including input devices and output devices, such as an LCD display, speaker, vibration generator, light, or other output device which may be used to communicate the detection of a change in neurochemical levels.

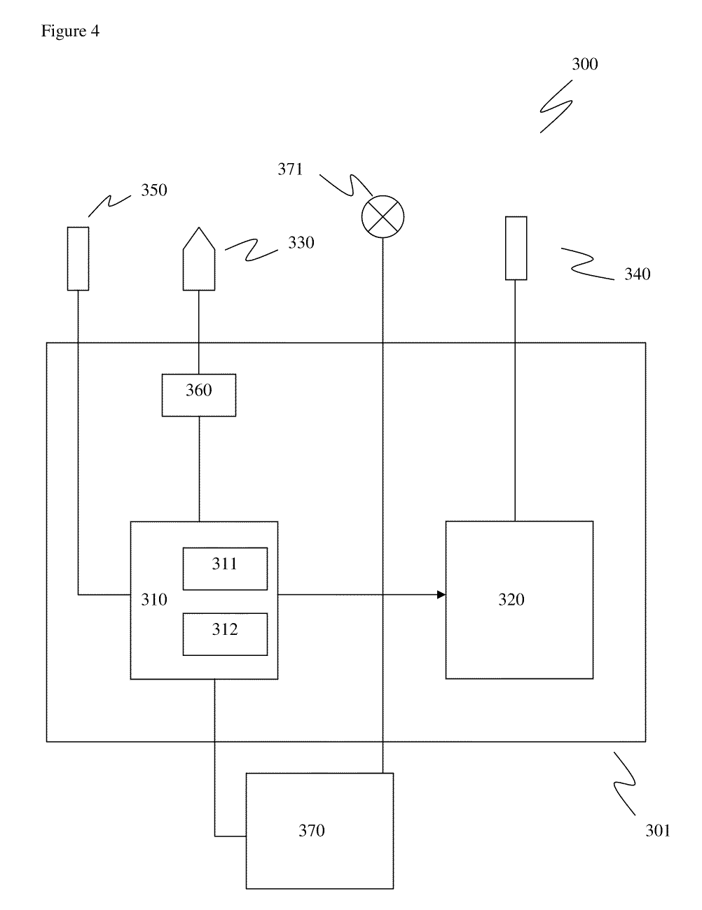

FIG. 4 shows a more detailed block diagram of the deep brain stimulator 300. FIG. 4 illustrates additional components, such as an amplification and conversion device 360 and chemical delivery module 370, that may be included. Amplification and conversion device 360 may be interposed between sensor 330 and control module 310. There are a number of commercial vendors who provide devices suitable for amplification, filtering, and analog/digital conversion of electrical signals representing oxidation/reduction currents obtained by sensor 330. In general, amplification and conversion device 360 should be capable of, but is not limited to, at least <20 ms waveform sampling, it should have, but is not limited to, at least 4 channel inputs for sensor 330 and electrode 340 (and can have up to 16, 32, and 128 inputs), and it should have analog or digital inputs and outputs. Amplification and conversion device 360 should be able to interface with other possible components of the deep brain stimulation device 300, including control module 310, and electrochemical processor 312. In particular, amplification and conversion device 360 should have an independently adjustable gain for each channel that is adjustable across a small range such as a maximum of 200,000 and a minimum of 50.

For example, an amplifier and conversion device 360 of the present invention will comprise an amplifier which has specifications, examples of which are outlined in, but are not limited to, those shown in Table 2:

TABLE 2
Amplifier Specifications

| Parameter | Value |
|---|---|
| Input Impedence | >200 MΩ/25 pF; |
| Sensitivity | 1 V/20 μV-1 V/10 mV |
| High Frequency | 100 Hz to 15 Hz in 8 steps, 6 dB/octave |
| filter | |
| Low Frequency | 0.5 Hz to 500 Hz in 8 steps, 6 dB/octave |
| filter | |
| Notch filter | >30 dB down at 60 Hz |
| CMRR | >100 dB at 60 Hz |
| Noise | <1 micro Volt rms from 2 Hz-10 kHz |
| | with input shortened |
| Calibration | 100 Hz squarewave, 2 μV/div to 10 mV/ |
| | div in 12 steps |
| Temperature | 20° C.-40° C. |
| measurement | |

The amplification and conversion device 360, in addition to being capable of amplifying an electrical signal, may be able to convert an analog oxidation/reduction current signal to a digital signal for transmission of the signal to the control module 310 and electrochemical processor 312. Amplification and conversion device 360 may also be capable of converting a digital signal to an analog signal. Methods and mechanisms for the conversion of analog to digital and digital to analog are well known to those of skill in the art and may be readily incorporated into an amplification and conversion device 360.

The components of stimulator 300 shown in FIG. 4 may be arranged such that they are in one housing or remote from one another. The components of stimulator 300 may be connected by means of wire, coaxial cable, optical cable, fiber optics, or infrared signals. Alternatively, several or all of the components of stimulator 300 may be in such close spatial proximity that they are connected by solder, other electrically conductive weld, or as part of a printable circuit. The components of stimulator 300 may be incorporated into a single device 301 by way of a housing, case, shell, frame, or other suitable mechanism, packaging, or confinement known to those of skill in the art. Packaged device 301 may be worn externally, such as on a belt-clip, harness, or lanyard, or may be implanted, such as under the skin of the chest, back, neck, or abdomen. Device 301 or components thereof are connected to electrodes 330 and 340 in the brain by means of wire, coaxial cable, or optical cable.

The present invention is based, in part, on the discovery that application of high frequency stimulation to the brain of an individual displaying symptoms of neurodegenerative disease with attendant alterations in levels of a neurochemical, such as Parkinson's disease, ameliorates the symptoms of the disease, and triggers the release of neurochemicals in, for example, the caudate-putamen. In particular, deep brain stimulation of the STN and/or MFB has been shown to stimulate the release of dopamine in the striatum of the rat and caudate-putamen of the monkey. Without being bound to one particular theory, it is believed that increased dopamine release may be the underlying mediator of deep brain stimulation-induced abolition of Parkinson's disease symptoms. Accordingly, the invention can include, in addition to the high frequency stimulation system taught herein, a chemical delivery system for administering neuroactive compounds (e.g., dopamine, L-dopa, or other dopamine analogs) in response to specific neurochemical (e.g., dopamine) levels in the caudate-putamen.

Deep brain stimulator 300, as shown in FIG. 4, may also include chemical delivery module 370 directly or indirectly connected to control module 310. Upon detection of specific neurochemical levels, control module 310 may, in addition to sending a signal to stimulation module 320, also send a signal to chemical delivery module 370. Control module 310 may be programmed to trigger the release of neuroactive compounds using different patterns. In one such pattern, each time control module 310 sends a signal to stimulation module 320 to generate electrical stimulation, a signal is also sent to chemical delivery module 370, causing it to release a neuroactive compound. Alternatively, release of a neuroactive compound from chemical delivery module 370 may be regulated by control module 310 based on a particular dosing regimen prescribed by a physician. For example, detection of a change in neurochemical levels by control module 310 will only send 1, 2, 3, or 4 or more signals to chemical delivery module 370 in a given period (e.g., every 24, 48 or 72 hours, or one dose every 6, 12, or 24 hours). Chemical delivery module 370 preferably includes a reservoir capable of containing a neuroactive compound and a pump, or its equivalent. Upon receipt of an appropriate signal, chemical delivery module 370 delivers the chemical (i.e., via a pump) from the reservoir to delivery module 371. Delivery module 371 may be a needle, syringe, catheter or other tubing, which is implanted or removably placed in close proximity to the site at which delivery of the chemical is desired (e.g., the brain, or more specifically, the caudate-putamen). The pump of chemical delivery module 370 may be a peristaltic-type pump, a mini-osmotic-type pump (such as those available from Alzet, Cupertino, Calif.), or other physiologically appropriate pump known to those of skill in the art. The chemical delivery module 370 may be incorporated in a housing 301 that also includes control module 310, amplification and conversion device 360 and stimulation module 320. Alternatively, chemical delivery module may be remote from the other components of the stimulator 300. Housing 301 may be implanted in an individual or worn externally. In addition, chemical delivery module 370 can be implanted in the individual separately from housing 301. For example, chemical delivery module 370 may be implanted in the abdomen or under the skin of the chest, wherein a tube or catheter extends from chemical delivery module 370 to delivery module 371 which is on, in, or near the, for example, caudate-putamen of the individual. Alternatively, all the components of high frequency stimulator 300, including chemical delivery module 370, are worn externally.

As indicated above, chemical delivery module 370 can be directly or indirectly connected to control module 310 such that a signal from control module 310 triggers release of chemical from chemical delivery module 370 via delivery module 371. Alternatively, chemical delivery module 370 can be manually controlled by the individual using a switch or other device. In this mode, control module 310 triggers some output that may be perceived by the individual. For example, the control module 310 may issue a tone, light, vibration, or mild electronic shock to signal the detection of specific neurochemical levels (e.g., such as a change in levels of a neurochemical). After perceiving the signal produced by control module 310, the individual can choose whether to manually trigger the chemical delivery module such that neuroactive chemical is delivered to the brain of the individual.

Chemical delivery module 370 may be used to deliver to an individual any composition of interest, e.g., a neuroactive compound. Preferably, the neuroactive compound is chosen from the group of neurochemicals, neuropeptides, neuromodulators, neurochemicals, receptor agonists, receptor antagonists, ion channel blockers, ion channel activators, and calcium chelators. Neuroactive compounds selected from glutamate, GABA, serotonin, norepinephrine, and dopamine are preferred. Other neuroactive compounds are contemplated by the invention and may be included in chemical delivery module 370 as desired.

It will be appreciated by one of skill in the art that electrical isolation may be provided between components of the deep brain stimulator. For example, electrical isolation may be provided between stimulation module 320 and control module 310, and or between the control module 310 and amplification and conversion device 360. Electrical isolation may be achieved using methods or components known in the art such as optical isolation.

### EXAMPLES

### Example 1

Immediate and sustained changes in both extracellular activity of subthalamic nuclei (STN) neurons in vitro and striatal dopamine efflux (release) in vivo were recorded in the rat brain to test the hypothesis that STN high frequency stimulation (HFS) excites local neuronal activity and enhances dopaminergic neurotransmission.

### Materials and Methods

In Vitro Brain Slice Preparation

For the preparation of slices, 4-6 weeks old male or female Sprague-Dawley rats (Iowa State University Animal Facility, Ames, Iowa) were deeply anesthetized with sodium pentobarbital (30-40 mg/kg) and killed by decapitation. The forebrain was rapidly removed and the hemispheres were separated with a midline incision. Four hundred micron thick slices were cut in the sagittal plane using a vibratome (Leica, Wetzlar, Germany). During preparation of slices, the tissue was placed in a chilled solution (5° C.) in which NaCl was replaced with sucrose while the osmolarity was maintained at 307 mOsm. Slices were placed in an interface style recording chamber (Fine Sciences Tools, Foster City, Calif.), maintained at 35° C. and allowed to recover for at least two hours. The bathing medium contained (in mM) NaCl, 126; KCl, 2.5; MgSO₄, 1.2; NaH₂PO₄, 1.25; CaCl₂, 2; NaHCO₃, 26; dextrose, 10 and was aerated with 95% O₂, 5% CO₂ to a final pH of 7.4. For the first 20 minutes that the slices were in the recording chamber, the bathing medium contained an equal mixture of the normal NaCl and the sucrose-substituted solutions.

In Vitro Electrophysiological Recordings

Extracellular recordings were obtained using glass microelectrodes and an extracellular amplifier (FHC, Inc., Bowdoinham, Me.). Extracellular recording electrodes were pulled on a P-80 micropipette puller (Sutter Instruments, Novato, Calif.) from medium-walled glass electrodes (WPI, Sarasota, Fla.). Micropipettes were filled with the bathing medium solution. In addition, the location of the STN was confirmed through Golgi staining. Monophasic constant current stimulation (50-100 μsec pulse width; 10-500 μA amplitude; 10-200 Hz frequency) of the STN was accomplished by placing a concentric stimulating electrode within ˜200 μm of the extracellular recording electrode in the STN. The current and voltage outputs were digitized and recorded with Digidata (Axon Instruments, Union City, Calif.) and stored on a computer and on videotape. Records were analyzed using PClamp 9.1 (Axon Instruments, Union City, Calif.).

Surgery and Constant Potential Amperometry

Twenty six male hooded Wistar rats, weighing 300±50 g, were obtained from the Animal Resources Center, Adelaide (SA, Australia). Rats were housed in pairs and maintained at a constant room temperature (22±0.5° C.) with a 12 h light:12 h dark cycle (lights on at 08.00 h). Food and water were available ad libitum. Rats were anaesthetized with urethane (1.5 g/kg, i.p., Sigma-Aldrich, St. Louis, Mo.), supplemented 30 min later with 0.3 g/kg urethane i.p. and mounted in a stereotaxic frame (David Kopf Instruments, Tujunga, Calif., USA) with the incisor bar set at −3.3 mm. Body temperature was maintained at 36±0.5° C. with a temperature-regulated heating pad (TC-831, CWE, NY, USA). Prior to surgery, 0.5 ml of 20% lidocaine-HCl was injected under the skin overlaying the skull. A single concentric bipolar stimulating electrode (SNE-100, David Kopf Instruments, Tujunga, Calif., USA) was implanted in the left STN of each animal (interaural coordinates: AP +5.2 mm, ML +2.2 mm, and DV +1.8 mm; Paxinos and Watson, 1997). Stimulation of regions dorsal to the STN was achieved by moving the stimulation electrode 0.2-0.4 mm dorsomedial to the STN site. Carbon-fibre recording electrodes (Thornel Type P, Union Carbide, Pittsburgh, Pa., USA) with an active recording surface of 500 μm (length) by 10 μm (od) were constructed as previously described (Forster and Blaha, 2003, Eur J Neurosci 17:751-762). A new recording electrode was used for each animal and was implanted into the left striatum (coordinates: AP; +1.2 mm, ML; +2.4 mm, and DV −4.4 mm from dura). An Ag/AgCl reference and stainless steel auxiliary electrode combination was placed in contact with contralateral cortical tissue 4 mm posterior to bregma.

Amperometric recordings were made within a custom-made Faraday cage to increase the signal-to-noise ratio. Following implantation of all electrodes, a fixed positive potential (0.8 V) was applied to the recording electrode, and oxidation current monitored continuously (10,000 samples/s) with an electrometer (Powerlab system, ADlnstruments, Sydney, NSW, Australia) and filtered at 50 Hz (Forster and Blaha, 2003; Dommett et al., 2005, Science 307:1476-1479). STN stimulation was applied following at least 60 min of implantation of the recording electrode.

Electrical Stimulation

A series of 15 cathodal monophasic current (25-1600 μA) pulses (0.5 ms duration) were delivered at 30 sec intervals to the concentric bipolar stimulating electrode implanted in the STN at a frequency of 5-300 Hz using an optical isolator and programmable pulse generator (Iso-Flex/Master-8; AMPI, Jerusalem, Israel). Extended stimulation of the STN, and regions immediately dorsal to the STN consisted of 1000 monophasic current (300 μA) pulses at 50 Hz. In separate experiments, nomifensine (20 mg/kg), fluoxetine (20 mg/kg) and desipramine (20 mg/kg) were administered via systemic injection (Sigma-Aldrich, St. Louis, Mo.).

Amperometry Data Analysis

Pre-stimulation baseline amperometric currents were normalized to zero current values, and data points between 0.25 sec before and 1-6 sec after the onset of the stimulation train were extracted from the continuous record for further analysis. The effects of reuptake inhibitors were determined using within-subjects comparisons. Thus for each animal, six current values corresponding to six STN-evoked responses, recorded before and after drug administration, were initially determined. Each of these current values was obtained by calculating total oxidation current between the time point at which the last pulse of a 15-pulse train terminated (as evidenced by the stimulus artifact) and the time point at which the elicited oxidation current returned to pre-stimulation levels. The averages of these pre- and post-drug current values were then utilized to determine mean drug-elicited percentage changes in individual animals, and these percentage changes were subsequently averaged across animals for each of the reuptake inhibitors tested. Mean STN-elicited striatal oxidation currents were statistically compared before and after drug administration for each drug group using paired, two-tailed t-tests. An alpha level for these analyses was set at 0.01.

In Vivo Electrode Histology

Upon completion of each constant potential amperometric experiment, an iron deposit was made at the site surrounding the tip of the STN-stimulating electrode with DC current (100 μA for 10 s). A DC current of 1 mA for 1 s was passed through each recording electrode to mark its position in the striatum. Rats were then killed with a 0.5-ml cardial injection of urethane (3.45 g/ml). Brains were removed, immersed overnight in 10% buffered formalin containing 0.1% potassium ferricyanide and stored in 30% sucrose/10% formalin until sectioning. After fixation, 60-μm coronal sections were cut on a cryostat at 30° C. A Prussian Blue spot resulting from the redox reaction of ferricyanide marked the stimulation site. The placements of stimulating and recording electrodes were determined under a light microscope and recorded on representative coronal diagrams (Paxinos and Watson, 1997, The rat brain in stereotaxic coordinates, 3rd ed. San Diego, Calif.: Academic Press).

Monoclonal Antibody Stain to Dopa-Transporter

Monoclonal antibody to DOPA-transporter was used to determine the anatomical relationship between the STN and SNc axonal fibers of passage. For the preparation of STN brain slices, male or female ferrets (Mustela putorious furo; Marshall Farms; North Rose, New York), 2-4 months old, were deeply anesthetized with sodium pentobarbital (30-40 mg/kg) and killed by decapitation. The brain was removed and left overnight at 4° C. in 20% sucrose and phosphate-buffered saline (PBS, pH=7.4) solution. The following day, 20 μm thick sagittal sections were cut serially through the STN with a cryostat (Leica, Wetzlar, Germany). Sections were fixed to gelatinized slides with 10% neutral buffered formalin. Sections were then incubated for 30 min in blocking solution (0.1 M PBS, 0.3% Triton X-100 and 5.0% normal rabbit serum) and then incubated for 48 h at 4° C. in rat anti-DAT primary antibody (Chemicon, Temecula, Calif., USA, 1:4000 in PBS, 0.3% triton X-100 and 1.0% normal rabbit serum). This was followed by overnight incubation at 4° C. in a biotinylated secondary antibody (Rabbit, Anti-rat IgG, 1:500, Vector Laboratories, Burlingame, Calif., USA) and for 2 h in 1:5000 avidin peroxidase with 0.75% Triton X-100. Sections were then reacted with cobalt and nickel-intensified diaminobenzidine (DAB, Sigma-Aldrich, St. Louis, Mo.) for 30 min. Hydrogen peroxide (3.33 μl ml-1) was added to the DAB solution for a further 8 min. Rinses (4×10 min) in 0.1 M PBS were performed between each step. Sections were dehydrated in a series of graded ethanol solutions (50-100%) and cleared (Histosolve, Amber Scientific, Australia) before being cover-slipped with a polystyrene mounting medium.

### Results

STN Stimulation-Elicited Firing Activity: Extracellular STN Neuronal Recordings

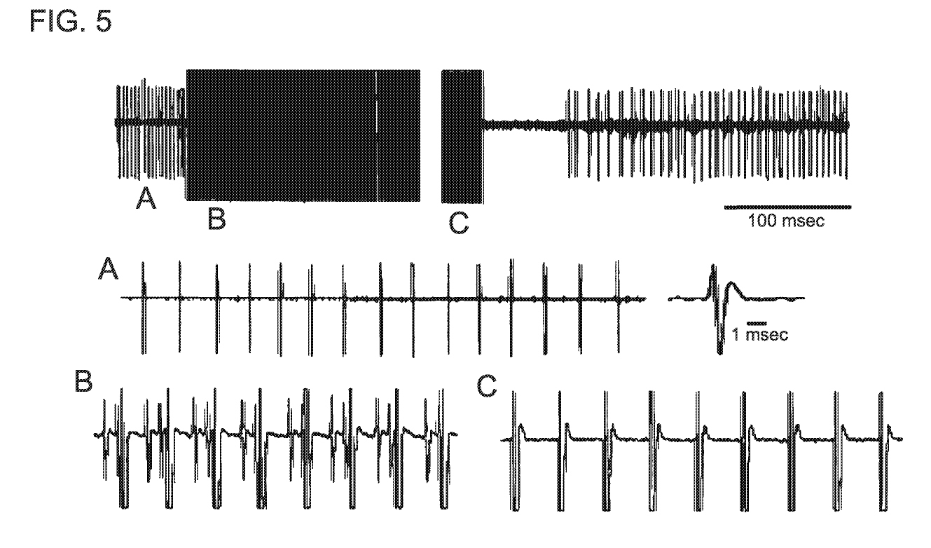

Using glass extracellular microelectrodes, unit recordings of STN neurons (n=7) were made before, during and after HFS of the STN (100 Hz for 10-30 seconds) with the stimulating electrode placed within ˜200 μm of the extracellular recording electrode. Prior to stimulation, the spontaneous STN neuronal firing rate was 24.9, 11.5 Hz. During the stimulation period, neuronal firing rate increased to 96.4, 29.6 Hz in the initial period of stimulation for approximately 2-5 seconds. As shown in FIG. 5, individual action potentials could be observed between the stimulus artifacts. Neuronal action potential firing eventually ceased despite continued stimulation. Upon termination of HFS, spontaneous neuronal firing returned to pre-HFS levels within 0.2 to 2 seconds.

STN Stimulation-Evoked Striatal Dopamine Efflux: Effects of Selective Reuptake Inhibitors

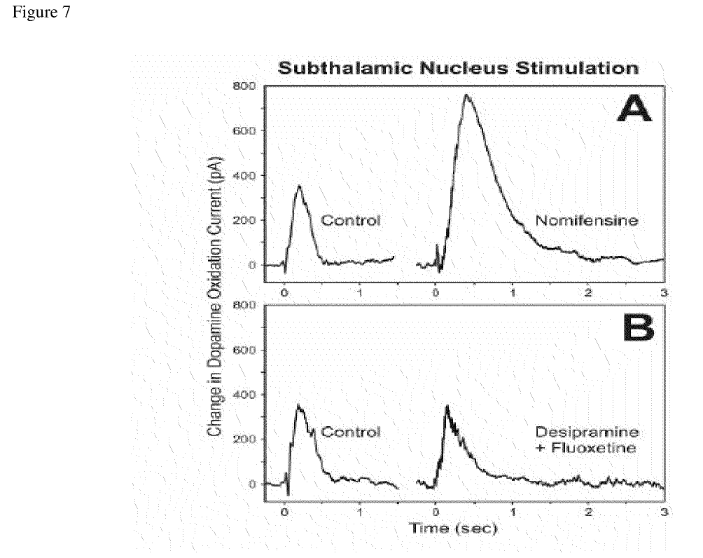

As shown in FIGS. 7A and B (Control), STN electrical stimulation (15 pulses, 50 Hz, 300 μA) evoked a rapid increase in striatal dopamine oxidation current (peak within 211±13 msec and amplitude of 350±14 pA) corresponding to dopamine efflux that was stimulus time-locked (Dugast et al., 1994). Upon cessation of stimulation, dopamine efflux rapidly returned to pre-stimulus levels within 577±22 msec of stimulation as a result of terminal dopamine reuptake (Suaud-Chagny et al., 1995; Suaud-Chagny, 2004). As shown in FIGS. 7A and B, systemic administration of the selective dopamine reuptake inhibitor (nomifensine; n=5 rat) significantly increased STN electrical stimulation-evoked dopamine oxidation current (765±38 pA; p<0.001 vs. pre-drug response) and delayed recovery to prestimulation baseline levels (2035±132 msec of stimulation; p<0.001 vs. predrug response). In contrast, combined systemic administration of the selective serotonin (fluoxetine) and norepinephrine (desipramine) reuptake inhibitors (n=4 rats) failed to significantly alter the pattern of dopamine oxidation current evoked by STN stimulation (330±12 pA vs. 342±9 pA within 215±7 msec vs. 209±11 msec and 615±18 msec vs. 630±28 msec total duration, pre vs. post-drug treatment, respectively).

STN Stimulation-Evoked Striatal Dopamine Efflux: Frequency and Intensity Dependence

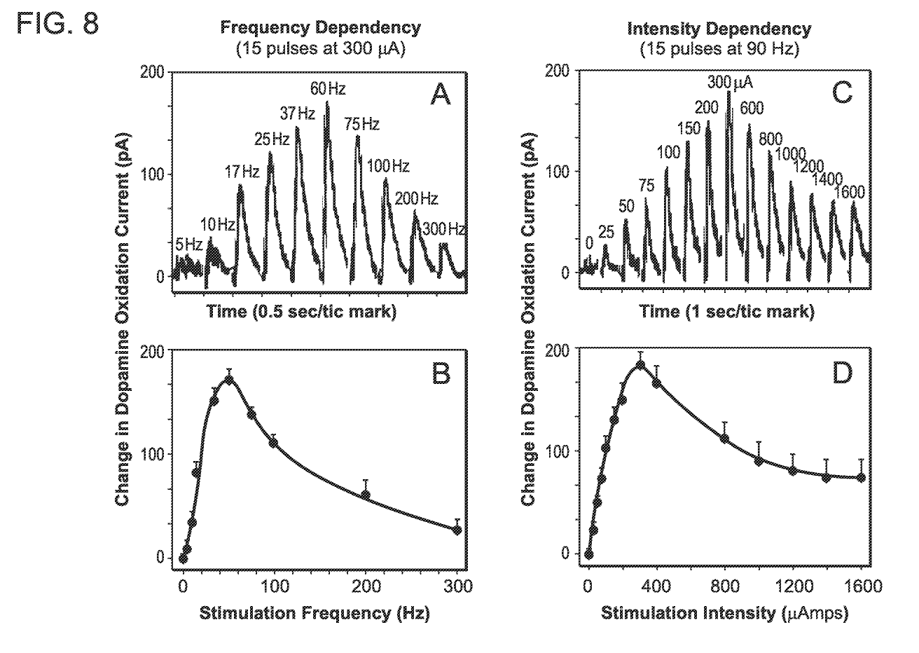

As shown in FIG. 8A, 15 pulses of 300 μA stimulation over a range of frequencies (5-300 Hz) resulted in a biphasic increase in striatal dopamine efflux, where maximal increases were observed between stimulation test frequencies of 37-75 Hz (n=6 rats). The relationship of maximal increases in dopamine efflux with respect to the applied test frequencies is illustrated in FIG. 8B.

As shown in FIG. 8C, 15 pulses of 50 Hz stimulation over a range of current intensities (25-1600 μA) also resulted in a biphasic increase in striatal dopamine efflux, where maximal increases were observed between stimulation test intensities of 200-600 μA (n=5 rats). The relationship of maximal increases in dopamine efflux with respect to the applied test intensities is illustrated in FIG. 8D.

Anatomical Relationship of STN and Dopaminergic Axonal Fibers of Passage

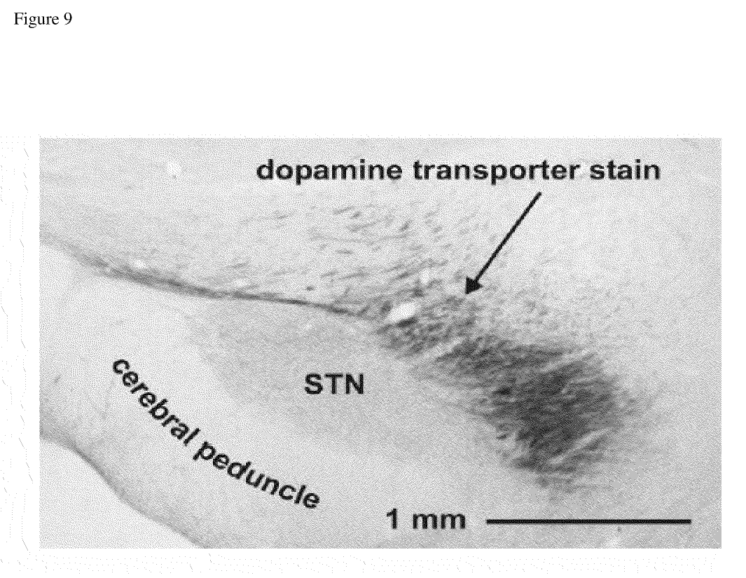

Dopaminergic fibers arising from dopaminergic neurons in the midbrain were observed to pass over the dorsal surface of the STN as identified using monoclonal antibody to the dopamine-transporter (FIG. 9; n=3 rats). A mediolateral gradient was apparent in the proportion of dopaminergic fibers running dorsal with respect to the coronal section of the core of the STN nucleus.

STN Stimulation-Evoked Striatal Dopamine Efflux: Regional Dependency

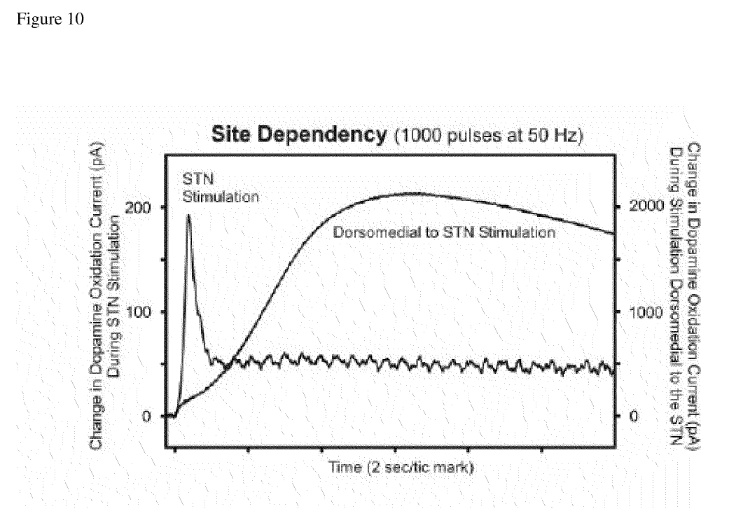

As shown in FIG. 10 (n=8 rats), extended duration of STN stimulation (1000 pulses, 300 μA, 50 Hz) evoked a brief increase in striatal dopamine efflux that was non-stimulus time-locked. A peak response in dopamine efflux (192±16 pA) was obtained within 365±14 msec, with a return to a sustained level of 52±8 pA above pre-stimulation baseline levels within 1030±35 msec, despite continuous application of the electrical stimulus. The duration of the initial increase in dopamine efflux closely corresponded to the duration of increased STN extracellular activity observed in slices of STN stimulated for prolonged periods. In marked contrast, similar stimulation of a region immediately dorsomedial to the STN resulted in an increase in striatal dopamine efflux that was stimulus time-locked and eleven fold greater in amplitude (2127±132 pA) than dopamine efflux after stimulation within the STN. The maximal increase was obtained within 6 sec and remained significantly elevated above baseline levels over the duration of the stimulation period.

### Conclusion

Enhanced dopamine release within the basal ganglia may be an important mechanism whereby deep brain stimulation ameliorates symptoms of Parkinson's disease. Deep brain stimulation of the STN transiently increased action potential firing in STN. Stimulation of ascending fibers dorsal to STN resulted in a greater and more prolonged release of striatal dopamine than STN stimulation. As such, deep brain stimulation of tissue immediately dorsal to the STN to optimally enhance dopamine release in the basal ganglia may prove to be an important mechanism whereby deep brain stimulation ameliorates symptoms of Parkinson's disease.

### Example 2

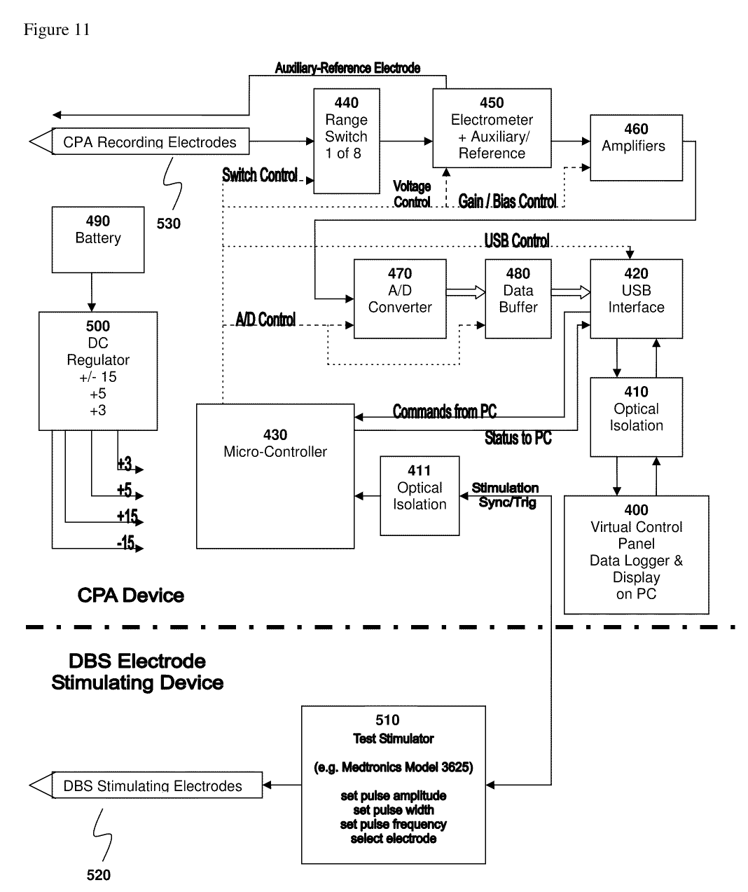

FIG. 11 shows a detailed example of a deep brain stimulator useful for positioning a stimulation electrode in the brain of an individual. Although the “CPA Recording Electrodes” and the “DBS Stimulating Electrodes” are depicted as separate electrodes, they can be combined as a single probe as described above.

Virtual control panel 400 comprises software on a conventional personal computer (PC) that provides control of the constant potential amperometry (CPA) device (neurochemical measurer and monitoring device). There may be a signaling device such as LED's (Light Emitting Diodes) on the CPA device to confirm activity and status, but no push buttons, keypads, LCD (Liquid Crystal Display) panels or rotary switches are necessary. The functionality of the CPA device is entirely controlled from the PC through Universal Serial Bus (USB) interface 420. The PC will show a graphical image of the CPA device and the various functions of the device (e.g., settings for DC power on-off, electrode potential, electrode selection, gain and amplification, etc.). In addition, the PC can serve as a graphics interface to display data recorded on-line. All data lines and command lines to and from the PC should be passed through optical isolation components to minimize any hazardous current flow from the alternating current (AC) power lines and the patient.

Optical isolation components 410 and 411 provide electrical isolation between the CPA Device, the PC and the deep brain stimulation (DBS) device. An optical isolator converts a pulse of current on the transmit side to a pulse of light. On the receiving side, the pulse of light is converted to a voltage pulse. Control and information is passed from one sub-system to another without physically connecting them with wires and thus hazardous currents being passed to the patient is avoided should an electronic failure occur.

USB interface 420 is a high speed serial interface with the PC. External computer devices can be connected to the PC via a simple serial interface cable and the installation procedures are user friendly (plug and play). In the case of the CPA device, digitized recording data and CPA device status data can pass from the CPA device to the PC for display. Control commands can pass from the PC to the CPA device to establish the proper data collection configuration. Note that the USB interface is optically isolated (410) from the PC to prevent hazardous currents from entering the patient from the AC power lines connected to the PC.

Micro-controller 430 receives commands from the PC (e.g., settings for DC power on-off, electrode potential, electrode selection, gain and amplification, etc.) via USB interface 420. The outputs of this component include “switch control”, “voltage control”, “gain/bias control”, “USB control”, “analog to digital (A/D) control”, and the CPA device “status to PC”. “Switch control” sets the range of current recorded from the “CPA recording electrodes” via range switch 440. “Voltage control” sets a constant potential (voltage) to the “auxiliary electrode” via the electrometer+auxiliary/reference 450. “Gain/bias control” sets the amplification parameters of amplifier 460. “USB control” monitors and sets data flow through the USB interface 420. “A/D control” monitors and sets the A/D converter 470 and accompanying data buffer 480. “Status to PC” provides system information from the CPA device and stimulus information from the DBS electrode stimulating device to be continuously monitored by the PC via USB interface 420.

Range switch 440 functions as an electronic switch that permits eight different current ranges to be selected by commands from the PC operating through micro-controller 430. Each setting determines the absolute range of current (e.g., 10 to 100 nanoamperes) that can be measured by the Electrometer 450 at any given time. The CPA recording electrodes 530 make electrical connection to the CPA device through range switch 440 which, in turn, makes electrical connection to electrometer 450. Although range switch 440 is shown as including eight ranges, range switch 440 can include any number of ranges. For example, it may not be necessary to have a 1 of 8 position range switch 440, but rather a 1 of 3 or 1 of 4.

Electrometer+auxiliary/reference 450 is a two or three-electrode high impedance current measurer and serves to measure current flow through the CPA recording electrodes 530 in tissue or aqueous solutions, via range switch 440. A constant potential (fixed voltage) is also provided to the “auxiliary/reference electrode” connected directly to electrometer 450. The analog output voltage (proportional to the input current to electrometer 450) is fed directly to amplifier 460.

Amplifiers 460 comprise circuitry that provides appropriate amplification of the analog output voltage at the output of electrometer+auxiliary/reference 450 circuits. This amplification is necessary to provide suitable voltage levels for the A/D converter 470 circuits. The gain and bias of these amplifier circuits are set as required to maintain signal fidelity by micro-controller 430.

A/D converter 470 serves to convert a voltage from the amplifier circuits of amplifiers 460 (proportional to the input analog current signal to electrometer 450) to a digital signal suitable for data processing. A/D converter 470 is under the control of the micro-controller 430. Digital signals from A/D converter 470 are fed into data buffer 480 for temporary storage.

Data buffer 480 serves to store and buffer the continuous flow of digital current signals from A/D converter 470 for on-line graphic display on the PC via USB interface 420. Data buffer 480 is under the control of micro-controller 430.

Battery 490 is a direct current (DC) battery that interfaces with DC regulator 500. DC regulator 500 serves as a voltage regulator to deliver power to the electronic units/components comprising the CPA device. This form of power supply minimizes any hazardous current from entering the patient from the AC lines supplying power to the PC.

Test stimulator 510 is connected to the DBS Stimulating Electrodes 520 and comprises any pre-existing (e.g., Medtronics 3625 test stimulator) or future electronic stimulation device used for DBS. The signal line “stimulation synchronization/triggering” connecting test stimulator 510 with micro-controller 430, via optical isolator 411, provides communication between the DBS electrode stimulating device and the CPA device. This communication may be uni- or bi-directional depending on the type of test stimulator employed. A minimal configuration will require uni-directional information of the timing and triggering of stimulation pulses from test stimulator 510 to the PC for the purpose of graphically presenting this information in synchronization with recorded digitized current data from the CPA Device. This signal will be optically isolated by optical isolation 411 to minimize any hazardous currents flowing into the patient from either of the two electronic devices.

Some of the blocks in the CPA device of FIG. 11 need not be as complex as shown. Likewise, the gain/bias control circuits may by optionally omitted.

### Example 3

Example 1 above demonstrates that high frequency stimulation (HFS) results in neurotransmitter release. In the present example, the hypothesis that HFS to thalamus or subthalamic nucleus (STN) induces astrocytic glutamate release capable of abolishing synchronized neural network oscillations was tested.

### Materials and Methods

In Vivo Glutamate Measurements in the Rat STN and Thalamus

The in vivo experiments were performed with male or female Sprague Dawley rats weighing an average of 250±55 grams. The rats were housed in plastic and steel cages in a temperature controlled room (21° C.) under a 12 hour light/12 hour dark cycle (light on at 08:00 hr). The rats had ad libitum access to food pellets and water prior to surgery. Before surgery, the rats were anaesthetised with ketamine (100 mg/mL) and xylazine (20 mg/mL). Once anaesthetized, the rats were placed in a Kopf stereotaxic frame in which the skull was secured with a nose clamp, incisor bar and ear bars. Constant body temperature (36.5° C.) was maintained using a heat pad grounded to an external source, and the animal's temperature was measured using a rectal thermometer. A 1.5-2 cm incision of the skin was made to expose the cranial landmarks of bregma and lambda. Coordinates for all electrode placements were obtained from the stereotaxic atlas of the rat's brain by Paxinos and Watson. After, a trephine hole was drilled over the left thalamus or STN to allow placement of the recording and stimulating electrodes.

In Vivo Electrode Histology

Upon completion of each in vivo experiment, a DC current of 1 mA for 1 s was passed through each recording electrode to mark its position. Rats were then killed by decapitation. Brains were removed, immersed overnight in 10% buffered formalin containing 0.1% potassium ferricyanide and stored in 30% sucrose/10% formalin until sectioning. After fixation, 60-μm coronal sections were cut on a cryostat at 30° C. A Prussian Blue spot resulting from the redox reaction of ferricyanide marked the stimulation site. The placements of stimulating and recording electrodes were determined under a light microscope and recorded on representative coronal diagrams.

Glutamate Electrochemistry

Glutamate biosensors (Pinnacle Technology Inc., Lawrence, Kans.) were manufactured as described by Hu et al. J. Neurochem. 1997 68:1745-1752. In brief, the sensor was made using lengths of Teflon-coated platinum iridium (7%) wire (Pt—Ir, 0.25 o.d., Medwire, Mount Vernon, N.Y.). A 0.05 mm Ag wire was wrapped on the Teflon coated Pt—Ir electrode and anodized to create an Ag/AgCl reference counter electrode. The sensing cavity was formed by stripping the Teflon coating from one end, revealing the bare Pt—Ir electrode (0.35 mm and 1.0 mm lengths). An interferent screening inner-membrane was fabricated on the bare Pt—Ir electrode. An enzyme layer was formed over the inner-membrane by co-immobilizing glutamate oxidase and ascorbate oxidase with glutaraldehyde and bovine serum albumin (BSA). Glutamate biosensors were tested in 0.1 M phosphate-buffered saline (PBS; 7.4) for a minimum glutamate sensitivity of 300 pA/uM and for insensitivity to ascorbate (response to 250 uM ascorbate less than 0.5 nA). Sensors that did not meet these criteria were rejected. Sensor lengths were manufactured for use with brain slices, with the electrode shaft at ˜15 mm with a sensing region of ˜350 um.

In Vitro Thalamic Slice Preparation

For the preparation of slices, 3-4 month old male or female ferrets (muftela putoriouf furo; Marshall Farms; North Rose, New York) were deeply anesthetized with sodium pentobarbital (30-40 mg/kg) and killed by decapitation. The forebrain was rapidly removed, and the hemispheres were separated with a midline incision. Four hundred micron thick slices were cut using a vibratome (Ted Pella, Inc.) in the sagittal plane. During preparation of slices, the tissue was placed in a solution (5° C.) in which NaCl was replaced with sucrose while maintaining an osmolarity of 307 mOsm to increase tissue viability. Slices were placed in an interface style recording chamber (Fine Sciences Tools) maintained at 34±1° C. and allowed at least two hours to recover. The bath was perfused with artificial cerebrospinal fluid (aCSF) which contained (in mM): NaCl, 126; KCl, 2.5; MgSO₄, 1.2; NaH₂PO₄, 1.25; CaCl₂, 2; NaHCO₃, 26; dextrose, 10 and was aerated with 95% O₂, 5% CO₂ to a final pH of 7.4. For the first 20 minutes of perfusion of the thalamic slices, the bathing medium contained an equal mixture of aCSF and the sucrose-substituted solution.

Electrophysiology

Intracellular recording electrodes were formed on a Sutter Instruments P-2000 laser micropipette puller from medium-walled glass (WPI, 1B100F). Micro-pipettes were filled with 2 M K-acetate. Only those neurons exhibiting a stable resting membrane potential of at least −60 mV and electrophysiological properties were included for analysis. Electrical stimulation was achieved through the placement of a concentric stimulating electrode and delivering stimulation (100 μsec duration; 10-500 μA amplitude; 100 Hz frequency). Mean values are given ±SEM. The data was analyzed using Chart (eDaq) on a Pentium style computer and figures were drawn using Core1DRAW (Corel).

Primary Astrocyte Culture

Astrocyte cultures were prepared from the cortices of neonatal rats (1-3 day old) using the Worthington Papain Dissociation System (Worthington Biochemical Corporation, Lakewood, N.J.). Briefly, cortices of neonatal rats were dissected, treated with papain (20 U/ml), dissociated by trituration and plated in 75 cm² flasks in Dulbecco's modified Eagle's medium supplemented with 10% charcoal-stripped FBS and 1% penicillin/streptomycin (100 U/ml penicillin, 100 μg/ml streptomycin). Cells were fed twice weekly until they reached confluence (Day 10-12 in vitro) at which point they were mechanically shaken for 1 hr on an orbital shaker to remove any remaining oligodendrocytes and microglia. Subsequently, cultures were treated with trypsin for 30 mins at 37° C., placed in an eppendorf tube and centrifuged at 100 g for 5 minutes. The cells were washed 2× in PBS prior to inserting the stimulating and glutamate recording electrodes into the cell pellet containing ˜2.0×10⁶ cells.

Immunocytochemistry

Astrocytes on coverslips were washed three times in PBS and then fixed in 4% paraformaldehyde for 10 minutes at room temperature. After rinsing again in PBS, coverslips were blocked in 10% normal goat serum for 30 mins followed by overnight incubation at 4° C. with primary antibody (mouse anti-rat GFAP, 1:500, GA5 clone, Sigma). The following day, slides were washed in PBS and secondary antibody was applied for 2 hours (goat anti-mouse Alexa Fluor 488 or 555, 1:250, Molecular Probes). After a final wash, cells were post-fixed in acid-alcohol (95% ethanol, 5% glacial acetic acid) for 10 mins, rinsed and mounted with VectaShield (Vector Laboratories), examined with an Olympus fluorescence microscope, and images were captured with a Q-Fire cooled camera.

### Results

Effect of HFS on Glutamate Release in the Thalamus and STN In Vivo

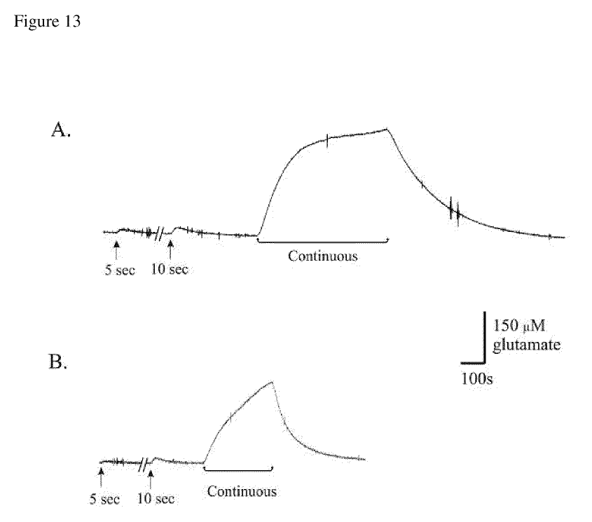

To test the hypothesis that glutamate was the neurotransmitter released during HFS, the extracellular glutamate concentration was measured using a dual enzyme-based electrochemical sensor in the STN and the ventrolateral (VL) thalamus of the rat in vivo. The concentric bipolar stimulating electrode and the glutamate sensor electrode were positioned within ˜200 μm of each other in the STN and VL thalamus in the anaesthetized rat placed in a Kopf stereotactic frame. HFS of the STN (100 Hz, 100 μs pulse width, 300 μA) resulted in an increase in extracellular glutamate in the STN (FIG. 13A; n=13). A similar increase in glutamate level was measured in the thalamus when HFS was delivered to the VL thalamus (FIG. 13B; n=10). Additionally, continuous stimulation of the STN or VL thalamus resulted in an immediate elevation of the glutamate level that remained elevated for the duration of the stimulation. Upon cessation of stimulation, the glutamate level slowly returned to pre-stimulation baseline. The correct placements of stimulating and recording electrodes in the STN or VL thalamus were confirmed under a light microscope in the sectioned rat brains.

HFS Effects on Synchronized Oscillations

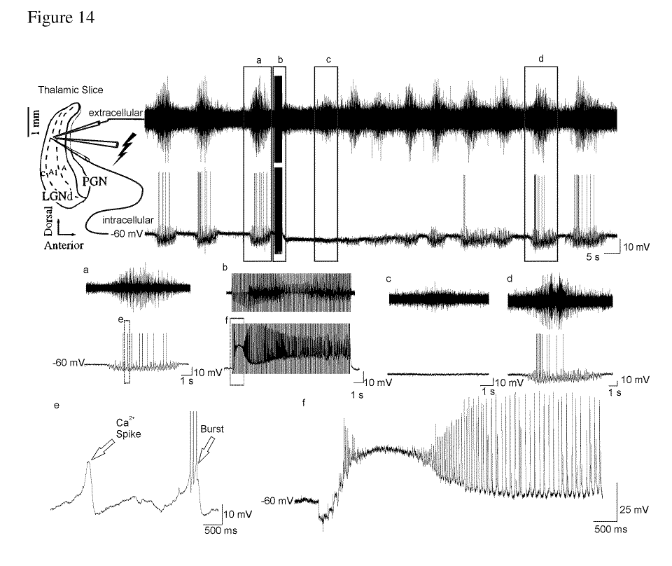

To test the functional effects of HFS-mediated glutamate release, simultaneous intracellular and extracellular recordings were made in the ferret lateral geniculate nucleus (LGN) in vitro slice preparation, which generates spontaneous spindle waves (FIG. 14; n=21). Spindle activity was recorded from a population of neurons (extracellular electrode), and the electrophysiological activity associated with the spindle activity was recorded from single neurons within that population. The intracellular recording revealed synchronized Ca²⁺ bursts (FIG. 14a, lower trace and FIG. 14e) concurrently with the population spindles (FIG. 14a, upper trace). HFS was applied by a stimulating electrode positioned within ˜100 μm of the intracellular and extracellular recording electrodes. It was not possible to observe the effect of HFS on spindle activity during HFS due to the stimulation artifact in the extracellular trace (FIG. 13b, upper trace). However, intracellularly, the stimulation artifact did not prevent the observation of an initial IPSP followed by a prolonged EPSP, membrane depolarization, action potential generation, depolarization block and further action potential generation (FIG. 14f; n=21). In the immediate post-stimulation period, neuronal activity was absent (FIG. 14c). The activity returned gradually in approximately 10-30 seconds while spindling returned in 30-60 seconds (FIG. 14d; n=21), indicating that the neurons were not lesioned or damaged (FIG. 14d).

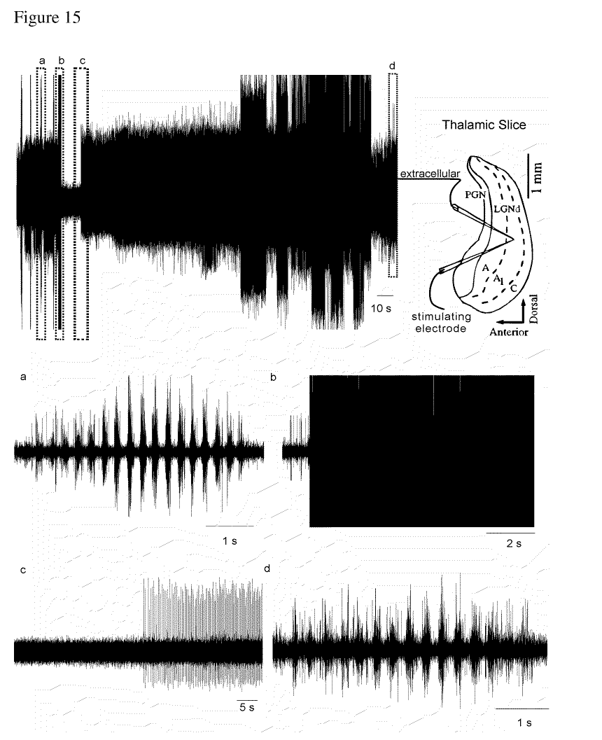

When GABA_{A} receptors were blocked by picrotoxin (20 μM), the spindle waves were transformed into events that resemble absence-seizure-like activity (FIG. 15; n=5 slices). In the picrotoxin-treated slices, HFS applied to thalamocortical relay neurons eliminated the 3-4 Hz seizure-like activity in 5 slices, as observed using extracellular recording electrodes (FIG. 15). Thus, HFS abolished both normal spontaneous synchronized oscillations, such as spindle waves, and abnormal oscillations such as, absence-seizure like 3 Hz oscillations. FIG. 15(a) shows an enlargement of an extracellular recording of a spontaneous slowed oscillation. FIG. 15(b) shows an enlargement of an extracellular recording during the stimulation period showing the stimulation artifact. FIG. 15(c) shows an enlargement of a portion showing the return of tonic action potential firing after a period of silence. FIG. 15(d) shows the reappearance of the slowed oscillations.

Amperometry/Glutamate Electrode

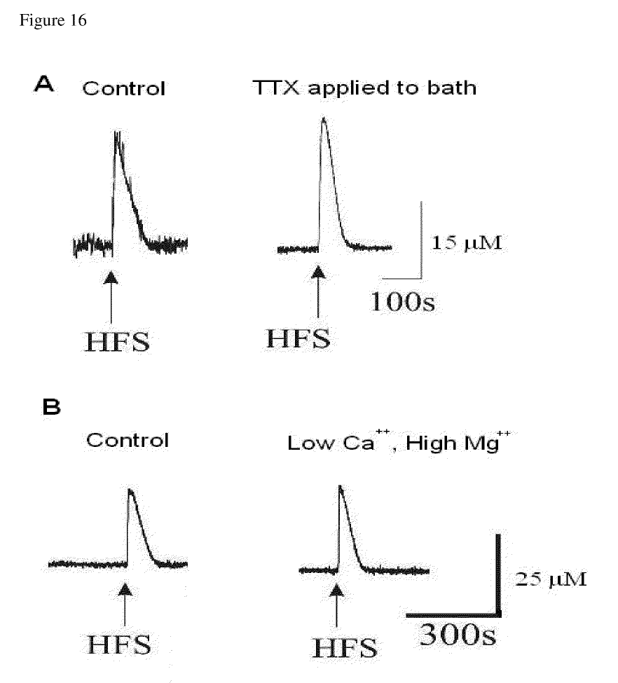

To confirm that glutamate was in fact responsible for the functional consequences of HFS, the extracellular glutamate concentration was also measured in the ferret thalamic slices in vitro using a glutamate sensor. The stimulating electrode and the glutamate sensor electrode were positioned within ˜100 μm of each other and placed in the A1 lamina of the LGN. During HFS (100 Hz, 100 μs pulse width, 300 μA), an increase in extracellular glutamate levels was measured in the control solution (FIG. 16, control; n=10) that was similar in characteristic to the increase measured in the rat in vivo (FIG. 13). To block the neuronal release of neurotransmitters, we used the Na⁺ channel blocker tetrodotoxin (FIG. 16A, TTX) or a high Mg++, low Ca++ solution (FIG. 16B). However, these classic neuronal exocytosis inhibitors failed to block the glutamate release induced by HFS.

Amperometry/Glutamate Electrode in the Primary Astrocytic Cultures

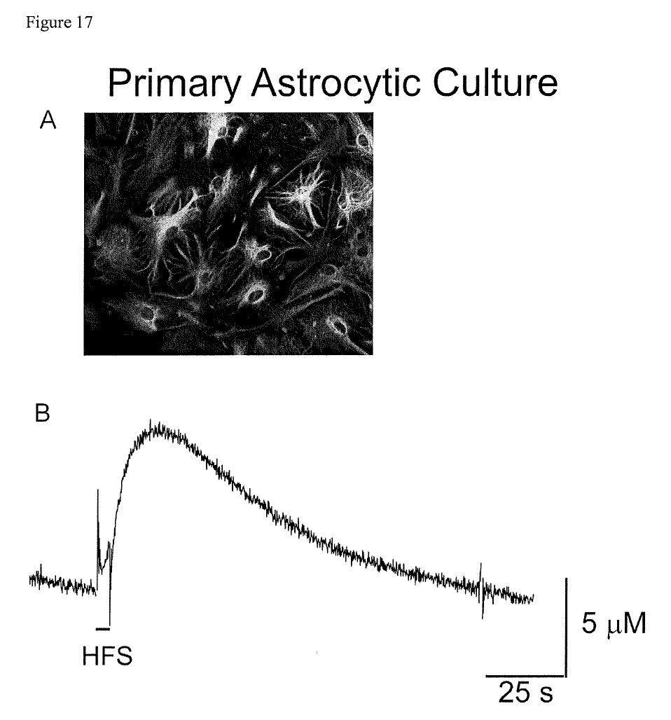

To test whether the glutamate released from HFS was of astrocytic origin, we utilized a primary astrocytic culture that was >98% pure as determined by glial fibrillary acidic protein (GFAP), a marker for astrocytes (FIG. 17A). HFS (10s duration, 100 Hz, 100 μs pulse width, 300 μA) of the purified astrocytes with the stimulating electrode and the glutamate sensor electrode positioned within ˜100 μm of each other, resulted in an increase in extracellular glutamate as measured by an enzyme-linked glutamate sensor. The glutamate level decreased to baseline upon cessation of stimulation. Of note, the HFS evoked glutamate release profile was similar to the glutamate release profile observed in the rat in vivo and in the ferret thalamic slices in vitro.

### CONCLUSION

These results suggest that HFS of the thalamus or STN leads to glutamate release from astrocytes that is insensitive to classic neuronal exocytosis inhibitors. HFS leads to astrocytic glutamate release and is able to abolish both normal spindle oscillations and abnormal 3 Hz absence-seizure-like oscillations. Thus, astrocytic glutamate release may be an important mechanism by which DBS is able to block abnormal neural network oscillations such as those that may be generated in tremor and seizures.

Other embodiments will be evident to those of skill in the art. It should be understood that the foregoing detailed description is provided for clarity only and is merely exemplary. The spirit and scope of the present invention are not limited to the above examples. All patents and publications cited herein are incorporated by reference in their entirety.

1. A method for modulating the levels of a neurochemical in the brain of an individual comprising:
(a) determining an amount of a neurochemical in a first brain region;
(b) determining whether said amount of said neurochemical is different from a predetermined amount; and
(c) if said amount of said neurochemical is different from said predetermined amount, applying electrical stimulation to a second brain region of said individual.

2. The method of claim 1, wherein said first and second brain regions are different regions of the brain.

3. The method of claim 1, wherein said first and second brain regions are the same brain region.

4. A method for modulating levels of a neurochemical in the brain of an individual comprising:
(a) providing a stimulation system comprising a neurochemical sensor, a control module, and a stimulation module under the control of said control module;
(b) placing said neurochemical sensor in a first brain region of said individual;
(c) placing an electrode operably connected to said stimulation module in a second brain region of an individual comprising neural tissue;
(d) determining the amount of said neurochemical in said first brain region using said sensor, and sending a signal indicative thereof to said control module;
(e) said control module performing the step of determining if the amount of said neurochemical is different from a predetermined amount, wherein if the amount of said neurochemical is different from said predetermined amount, sending a signal to said stimulation module;
(f) said stimulation module sending electrical stimulation to said electrode in said second brain region, whereby the levels of said neurochemical are modulated or regulated.

5. The method of claim 4, wherein steps (d) through (f) are repeated until said amount of said neurochemical in said first brain region of said individual is equal to said predetermined amount.

6. The method of claim 4, wherein said first brain region is the caudate-putamen.

7. The method of claim 4, wherein said second brain region is the subthalamic nucleus.

8. The method of claim 7, wherein said first brain region is a terminal targets of the nigrostriatal tract.

9. The method of claim 4, wherein said neurochemical is selected from the group consisting of dopamine, acetylecholine, glutamate, norepinephrine, epinephrine, serotonin, L-DOPA, and DOPAC.

10. The method of claim 10, wherein said neurochemical is dopamine.

11. The method of claim 4, wherein said second brain region is the medial forebrain bundle.

12. The method of claim 4, wherein said sensor is an electrochemical sensor.

13. The method of claim 13, wherein said electrochemical sensor is a carbon-based electrode.

14. The method of claim 4, wherein said sensor and said electrode are both present on a single probe.

15. A method for treating Parkinson's disease comprising:
(a) providing a stimulation system comprising a sensor, a control module, and a stimulation module under the control of said control module;
(b) placing an electrode operably connected to said stimulation module in the subthalamic nucleus or medial forebrain bundle of an individual having Parkinson's disease;
(c) placing said sensor in the caudate-putamen of said individual;
(d) determining an amount of dopamine in the caudate-putamen of said individual using said sensor and sending a signal indicative thereof to said control module;
(e) said control module performing the step of determining if said amount of dopamine is different from a predetermined amount, wherein if said amount of dopamine is different from said predetermined amount, sending a signal to said stimulation module; and
(f) said stimulation module sending electrical stimulation to said electrode, whereby Parkinson's disease is treated.

16. The method of claim 15, wherein said step of determining the amount of dopamine in the caudate-putamen is performed by constant potential amperometry.

17. The method of claim 16, wherein said sensor is an electrochemical sensor.

18. The method of claim 16, wherein steps (d) through (f) are repeated until said amount of dopamine in the caudate-putamen of said individual is equal to said desired amount.

19. The method of claim 16, wherein said electrode is placed in the subthalamic nucleus.

20. The method of claim 16, wherein said electrode is placed in the nigrostriatal tract.

21. The method of claim 16, wherein said electrode is placed in the medial forebrain bundle.

22. The method of claim 16, wherein said sensor and said electrode are present on a single probe.

23. A method for positioning a stimulation electrode in the brain of an individual comprising:
(a) providing a stimulation system comprising a sensor, a control module, and a stimulation module under the control of said control module;
(b) placing said sensor in a first brain region of said individual;
(c) placing a stimulation electrode operably connected to said stimulation module in a second brain region of an individual comprising neural tissue;
(d) applying an electrical stimulation signal to said stimulation electrode;
(e) determining the amount of a neurochemical in said first brain region using said sensor, and sending a signal indicative thereof to said control module;
(f) said control module performing the step of determining if the amount of said neurochemical reaches a predetermined amount;
wherein if the amount of said neurochemical does not reach said predetermined amount, the placement of said stimulation electrode is changed and steps (d)-(f) are repeated until a placement of the stimulation electrode is achieved in which said electrical stimulation results in an amount of said neurochemical which reaches said predetermined amount.

24. The method of claim 23, wherein said sensor and said stimulation electrode are both present on a single probe.

25. A deep brain stimulator comprising:
a sensor;
a control module operably connected to said sensor, wherein said control module is adapted to measure an electrochemical signal detected by said sensor;
a stimulation module operably connected to said control module; and
a stimulating electrode operably connected to said stimulation module; wherein said control module is adapted to determine the amount of a neurochemical in a brain region of an individual, wherein if the amount of said neurochemical differs from a predetermined amount, said control module being further adapted to send a signal to said stimulator to generate electrical stimulation.

26. The deep brain stimulator of claim 25, wherein said sensor and said stimulating electrode are both present on a single probe.
